(19) United States
(12) Patent Application Publication
(10) Pub. No.: US 2014/0220690 A1
(43) Pub. Date: Aug. 7, 2014

(54) MAMMALIAN GENES INVOLVED IN INFECTION

(71) Applicants: Donald Rubin, Nashville, TN (US); Thomas Hodge, Athens, GA (US); Robert Jeffrey Hogan, Watkinsville, GA (US)

(72) Inventors: Donald Rubin, Nashville, TN (US); Thomas Hodge, Athens, GA (US); Robert Jeffrey Hogan, Watkinsville, GA (US)

(73) Assignee: VANDERBILT UNIVERSITY, Nashville, TN (US)

(21) Appl. No.: 13/877,247

(22) PCT Filed: Oct. 28, 2010
(86) PCT No.: PCT/US10/54568

§ 371 Date: Mar. 31, 2014

Related U.S. Application Data
(60) Provisional application No. 61/388,657, filed on Oct. 1, 2010.

(30) Foreign Application Priority Data
Nov. 11, 2009 (US) 12616157

(51) Int. Cl. C12N 15/85

(52) U.S. Cl. CPC C12N 15/85
USPC 435/455; 435/325

(57) ABSTRACT

The present invention relates to nucleic acid sequences and cellular proteins encoded by these sequences that are involved in infection or are otherwise associated with the life cycle of one or more pathogens.

### CROSS-REFERENCE TO RELATED APPLICATION

This application claims priority to U.S. Provisional Application 61/388,657, filed Oct. 1, 2010, both of which are hereby incorporated by reference in their entirety.

### FIELD OF THE INVENTION

The present invention relates to nucleic acid sequences and cellular proteins encoded by these sequences that are involved in infection or are otherwise associated with the life cycle of one or more pathogens, such as a virus, a bacteria, a fungus or a parasite. The invention also relates to modulators of nucleic acid sequences and cellular proteins encoded by these sequences that are involved in infection or are otherwise associated with the life cycle of a pathogen.

### BACKGROUND

Infectious diseases affect the health of people and animals around the world, causing serious illness and death. Black Plague devastated the human population in Europe during the middle ages. Pandemic flu lulled millions of people in the 20^{th} century and is a threat to reemerge.

Viruses, which interfere with normal cellular processes, cause some of the most feared, widespread, and devastating human diseases. These include influenza, poliomyelitis, smallpox, Ebola, yellow fever, measles and AIDS, to name a few. Viruses are also responsible for many cases of human disease including encephalitis, meningitis, pneumonia, hepatitis and cervical cancer, warts and the common cold. Furthermore, viruses causing respiratory infections, and diarrhea in young children lead to millions of deaths each year in less-developed countries. Also, a number of newly emerging human diseases such as SARS are caused by viruses. In addition, the threat of a bioterrorist-designed pathogen is ever present.

While vaccines have been effective to prevent certain viral infections, relatively few vaccines are available or wholly effective, have inherent risks and tend to be specific for particular conditions. Vaccines are of limited value against rapidly mutating viruses and cannot anticipate emerging viruses or new bioterrorist designed viruses. Currently there is no good answer to these threats.

Traditional treatments for viral infection include pharmaceuticals aimed at specific virus derived proteins, such as HIV protease or reverse transcriptase, or the administration of recombinant (cloned) immune modulators (host derived), such as the interferons. However, the vast majority of viruses lack an effective drug. Those drugs that exist have several limitations and drawbacks that including limited effectiveness, toxicity, and high rates of viral mutations which render antiviral pharmaceuticals ineffective. Thus, an urgent need exists for alternative treatments for viruses and other infectious diseases, and methods of identifying new drugs to combat these threats.

### SUMMARY OF THE INVENTION

The present invention provides AZIN1, CENPL, C6orf111 (SFRS18), INHBA, NAV3, ODZ2, Ost-alpha, Ost-beta, PSMA4, RHOA, RPL28, RPL3, SFRS3, SYNGR1 and TXNRD1 nucleic acid sequences and proteins encoded by these sequences that are involved in infection by one or more pathogens such as a virus, a parasite, a bacteria or a fungus, or are otherwise associated with the life cycle of a pathogen. Also provided are methods of decreasing infection in a cell by a pathogen comprising decreasing expression or activity of one or more of AZIN1, CENPL, C6orf111 (SFRS18), INHBA, NAV3, ODZ2, Ost-alpha, Ost-beta, PSMA4, RHOA, RPL28, RPL3, SFRS3. SYNCR1 and TXNRD1. Also provided are methods of decreasing infection by a pathogen in a subject by administering an agent that decreases the expression and/or activity of AZIN1. CENPL, C6orf111 (SFRS18), INHBA, NAV3, ODZ2, Ost-alpha, Ost-beta, PSMA4, RHOA, RPL28, RPL3, SFRS3, SYNCR1 and TXNRD1. Further provided are methods of identifying an agent that decreases infection by a pathogen.

### DETAILED DESCRIPTION OF THE INVENTION

The present invention may be understood more readily by reference to the following detailed description of preferred embodiments of the invention and the Examples included therein.

Before the present compounds, compositions and/or methods are disclosed and described, it is to be understood that this invention is not limited to specific nucleic acids, specific polypeptides, or to particular methods, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. For example, the phrase “optionally obtained prior to treatment” means obtained before treatment, after treatment, or not at all.

As used throughout, by “subject” is meant an individual. Preferably, the subject is a mammal such as a primate, and, more preferably, a human. Non-human primates include marmosets, monkeys, chimpanzees, gorillas, orangutans, and gibbons, to name a few. The term “subject” includes domesticated animals, such as cats, dogs, etc., livestock (for example, cattle, horses, pigs, sheep, goals, etc.), laboratory animals (for example, ferret, chinchilla, mouse, rabbit, rat, gerbil, guinea pig, etc.) and avian species (for example, chickens, turkeys, ducks, pheasants, pigeons, doves, parrots, cockatoos, geese, etc.). The subjects of the present invention can also include, but are not limited to fish (for example, zebrafish, goldfish, tilapia, salmon and trout), amphibians and reptiles.

AZIN1, CENPL, C6orf111 (SFRS18), INHBA, NAV3, ODZ2, Ost-alpha, Ost-beta, PSMA4, RHOA, RPL28, RPL3, SFRS3, SYNGR1 and TXNRD1, host genes involved in viral infection, were identified using gene trap methods that were designed to identify host genes that are necessary for viral infection or growth, but nonessential for cellular survival. These gene trap methods are set forth in the Examples as well as in U.S. Pat. No. 6,448,000 and U.S. Pat. No. 6,777,177. U.S. Pat. Nos. 6,448,000 and 6,777,177 and are both incorporated herein in their entireties by this reference.

As used herein, a gene “nonessential for cellular survival” means a gene for which disruption of one or both alleles results in a cell viable for at least a period of time which allows viral replication to be decreased or inhibited in a cell. Such a decrease can be utilized for preventative or therapeutic uses or used in research. A gene necessary for pathogenic infection or growth means the gene product of this gene, either protein or RNA, secreted or not, is necessary, either directly or indirectly in some way for the pathogen to grow. As utilized throughout, “gene product” is the RNA or protein resulting from the expression of a gene.

The nucleic acids set forth herein and their encoded proteins can be involved in all phases of viral life cycles including, but not limited to, viral attachment to cellular receptors, viral infection, viral entry, internalization, disassembly of the virus, viral replication, genomic integration of viral sequences, transcription of viral RNA, translation of viral mRNA, transcription of cellular proteins, translation of cellular proteins, trafficking, proteolytic cleavage of viral proteins or cellular proteins, assembly of viral particles, budding, cell lysis and egress of virus from the cells. Although AZIN1, CENPL, C6orf111 (SFRS18), INHBA, NAV3, ODZ2, Ost-alpha, Ost-beta, PSMA4, RHOA, RPL28, RPL3, SFRS3, SYNGR1 and TXNRD1 were identified as cellular genes involved in viral infection, as discussed throughout, the present invention is not limited to viral infection. Therefore, any of these nucleic acid sequence and the proteins encoded by these sequences can be involved in infection by any infectious pathogen such as a bacteria, a fungus or a parasite which includes involvement in any phase, of the infectious pathogen's life cycle.

AZIN1. CENPL, C6orf111 (SFRS18), INHBA, NAV3, ODZ2, Ost-alpha, Ost-beta, PSMA4, RHOA, RPL28, RPL3, SFRS3, SYNGR1 and TXNRD1 are set forth in Table 1 as genes involved in viral infection. Additional identifying information for each of these genes is also set forth in Table 1. As utilized herein, when referring to any of the genes in this table, for example, and not to be limiting, AZIN1, this includes any AZIN1 gene, nucleic acid (DNA or RNA) or protein from any organism that retains at least one activity of AZIN1 and can function as an AZIN1 nucleic acid or protein utilized by a pathogen. For example, the nucleic acid or protein sequence can be from or in a cell in a human, a non-human primate, a mouse, a rat, a cat, a dog, a chimpanzee, a horse, a cow, a pig, a sheep, a guinea pig, a rabbit, a zebrafish, a chicken, to name a few.

As used herein, a gene is a nucleic acid sequence that encodes a polypeptide under the control of a regulatory sequence, such as a promoter or operator. The coding sequence of the gene is the portion transcribed and translated into a polypeptide (in vivo, in vitro or in situ) when placed under the control of an appropriate regulatory sequence. The boundaries of the coding sequence can be determined by a start codon al the 5′ (amino) terminus and a stop codon at the 3′ (carboxyl) terminus. If the coding sequence is intended to be expressed in a eukaryotic cell, a polyadenylation signal and transcription termination sequence can be included 3′ to the coding sequence.

Transcriptional and translational control sequences include, but are not limited to, DNA regulatory sequences such as promoters, enhancers, and terminators that provide for the expression of the coding sequence, such as expression in a host cell. A polyadenylation signal is an exemplary eukaryotic control sequence. A promoter is a regulatory region capable of binding RNA polymerase and initiating transcription of a downstream (3′ direction) coding sequence. Additionally, a gene can include a signal sequence at the beginning of the coding sequence of a protein to be secreted or expressed on the surface of a cell. This sequence can encode a signal peptide, N-terminal to the mature polypeptide, which directs the host cell to translocate the polypeptide.

Table 1 (column 2) provides one or more aliases for each of the genes set forth herein. Therefore, it is clear that when referring to a gene, this also includes known alias(es) and any aliases attributed to the genes listed in Table 1 in the future. The proteins encoded by the genes, if available, are also listed in column 3 of Table 1. In addition to the function of being involved in pathogenic infection as provided herein, a function of the proteins is also provided, if available, in column 4 of Table 1. The chromosomal location of the gene in the human genome (column 5) is also set forth. Thus, the present invention identifies a genomic loci of genes associated with viral infection. By identifying the gene and its location in the genome, the invention provides both the gene and its product(s) as targets for therapies such as antiviral, antibacterial, antifungal and antiparasitic therapies, to name a few.

Also provided in Table 1 are the GenBank Accession Nos. for the human mRNA sequences (column 6) and the GenBank Accession Nos. for the human protein sequences (column 7), if available. The nucleic acid sequences and protein sequences provided under the GenBank Accession Nos. mentioned herein are hereby incorporated in their entireties by this reference. One of skill in the art would know that the nucleotide sequences provided under the GenBank Accession Nos. set forth herein can be readily obtained from the National Center for Biotechnology Information at the National library of Medicine (http://www.ncbi.nlm.nih.gov/entrez/query.fcgi?db=nucleotide). Similarly, the protein sequences set forth herein can be readily obtained from the National Center for Biotechnology Information at the National Library of Medicine (http://www.ncbi.nlm.nih.gov/entrez/query.fcgi?db=protein). The nucleic acid sequences and protein sequences provided under the GenBank Accession Nos. mentioned herein are hereby incorporated in their entireties by this reference.

These examples are not meant to be limiting as one of skill in the art would know how to obtain additional sequences for the genes and gene products listed in Table 1 from other species by accessing GenBank or other sequence databases. One of skill in the art would also know bow to align the sequences disclosed herein with sequences from other species in order to determine similarities and differences between the sequences set forth in Table 1 and related sequences, for example, by utilizing BLAST. As set forth herein, a nucleic acid sequence for any of the genes set forth in Table 1 can be a full-length wild-type (or native) sequence, a genomic sequence, a variant (for example, an allelic variant or a splice variant), a nucleic acid fragment, a homolog or a fusion sequence that retains the activity of the gene utilized by the pathogen or its encoded gene product. For example, AZIN1 activity includes, but is not limited to, converting ornithine to putrescine as well as the ability to function as a cellular nucleic acid or protein involved in infection.

Further provided are the Entrez Gene numbers for the human genes (column 8). The information provided under the Entrez Gene numbers listed in Table 1 is also hereby incorporated entirely by this reference. One of skill in the art can readily obtain this information from the National Center for Biotechnology Information at the National Library of Medicine (http://www.ncbi.nlm.nih.gov/entrez/query.fcgi?db=gene). By accessing Entrez Gene, one of skill in the art can readily obtain additional information about every gene listed in Table 1, such as the genomic location of the gene, a summary of the properties of the protein encoded by the gene, information on homologs of the gene as well as numerous reference sequences, such as the genomic, mRNA and protein sequences for each gene. Thus, in addition to the sequences set forth under the GenBank Accession Nos. in Table 1, one of skill in the an can readily obtain additional sequences, such as genomic, mRNA and protein sequences by accessing additional information available under the Entrez Gene number provided for each gene. Thus, all of the information readily obtained from the Entrez Gene Nos. set forth herein is also hereby incorporated by reference in its entirety.

TABLE 1

| | | | | Human | Human GenBank | Human GenBank | |
|---|---|---|---|---|---|---|---|
| | | | | Chromosomal | Accession No. for | Accession No. for | |
| Gene | Alias | Definition | Known Cellular Functions | Location | mRNA | protein | Entrez Gene No. |
| AZIN1 | OAZI; OAZIN; | antizyme inhibitor 1 | Catalyzes the conversion of | 8q22.3 | NM_015878.4 | NP_056962.2 | 51582 |
| | ODC1L; MGC691; MGC3832 | | ornithine to putrescine in the | | NM_148174.2 | NP_680479.1 | |
| | | | first step in polyamine | | | | |
| | | | biosynthesis. Ornithine | | | | |
| | | | decarboxylase antizymes play a | | | | |
| | | | role in the regulation of | | | | |
| | | | polyamine synthesis by binding | | | | |
| | | | to and inhibiting ornithine | | | | |
| | | | decarboxylase. | | | | |
| CENPL | CENP-L; C1orf155; | centromere protein L | Component of a centromeric | 1q25.1 | NM_033319.1 | NP_201576.1 | 91687 |
| | FLJ31044; FLJ31786; | | complex involved in assembly | | | | |
| | dJ383J4.3; RP3-383J4.1 | | of kinetochore proteins. mitotic | | | | |
| | | | progression and chromosome | | | | |
| | | | segregation | | | | |
| C6orf111 (SFRS18) | HSPC306; SRrp130; | splicing factor, | The SR family proteins and SR- | 6q16.3 | NM_015491.1 | NP_056306.1 | 25957 |
| | C6orf111; FLJ14752; | arginine/serine-rich | related polypeptides are | | NM_032870.2 | NP_116259.2 | |
| | FLJ14853; FLJ14992; | 18 | important regulators of pre- | | | | |
| | FLJ90147; bA9819.2; | | mRNA splicing | | | | |
| | MGC104269; RP11- | | | | | | |
| | 9819.2; DKFZp564B0769 | | | | | | |
| INHBA | EDF; FRP | Inhibin, beta A | The inhibin beta A subunit | 7q15-p13 | NM_002192.2 | NP_002183.1 | 3624 |
| | | | joins the alpha subunit to form | | | | |
| | | | a pituitary FSH secretion | | | | |
| | | | inhibitor. Inhibin can be both a | | | | |
| | | | growth/differentiation factor | | | | |
| | | | and a hormone. Furthermore, | | | | |
| | | | the beta A subunit forms a | | | | |
| | | | homodimer, activin A, and also | | | | |
| | | | joins with a beta B subunit to | | | | |
| | | | form a heterodimer, activin AB, | | | | |
| | | | both of which stimulate FSH | | | | |
| | | | secretion. | | | | |
| NAV3 | POMFIL1; unc53H3; | neuron navigator 3 | This gene belongs to the neuron | 12q14.3 | NM_014903.4 | NP_055718.4 | 89795 |
| | KIAA0938; STEERIN3 | | navigator family and is | | | | |
| | | | expressed predominantly in the | | | | |
| | | | nervous system. The encoded | | | | |
| | | | protein contains coiled-coil | | | | |
| | | | domains and conserved AAA | | | | |
| | | | domain characteristic for | | | | |
| | | | ATPases associated with a | | | | |
| | | | variety of cellular activities. | | | | |
| | | | This gene is similar to unc-53, a | | | | |
| | | | Caenorhabditis elegans gene | | | | |
| | | | involved in axon guidance. | | | | |
| ODZ2 | TEN-M2; | odz, odd Oz/ten-m | May function as cellular signal | 5q34-q35.1 | NM_001122679.1 | NP_001116151.1 | 57451 |
| | DKFZp686A1568 | homolog 2 | transducer | | | | |
| | Tencurin-2 | (Drosophila) | | | | | |
| Ostalpha | OSTalpha; MGC39807 | organic solute | Able to mediate transport of | 3q29 | NM_152672.4 | NP_689885.3 | 200931 |
| | | transporter alpha | estrone 3-sulfate, | | | | |
| | | | dehydroepiandrosterone 3- | | | | |
| | | | sulfate, taurochlate, digoxin, | | | | |
| | | | and prostaglandin E2, | | | | |
| | | | indicating a role in the | | | | |
| | | | disposition of key cellular | | | | |
| | | | metabolites of signaling | | | | |
| | | | molecules. | | | | |
| Ostbeta | OSTbeta; FLJ26090; | organic solute | Able to mediate transport of | 15q22.31 | NM_178859.2 | NP_849190.1 | 123264 |
| | MGC118959; MGC118960; MGC118961 | transporter beta | estrone 3-sulfate, | | | | |
| | | | dehydroepiandrosterone 3- | | | | |
| | | | sulfate, taurochlate, digoxin, | | | | |
| | | | and prostaglandin E2, | | | | |
| | | | indicating a role in the | | | | |
| | | | disposition of key cellular | | | | |
| | | | metabolites of signaling | | | | |
| | | | molecules. | | | | |
| PSMA4 | HC9; PSC9; HsT17706; | proteasome | The proteasome is a | 15q25.1 | NM_001102667.1 | NP_001096137.1 | 5685 |
| | MGC12467; MGC24813; MGC111191 | (prosome, | multicatalytic proteinase | | NM_001102668.1 | NP_001096138.1 | |
| | | inacropain) subunit. | complex with a highly ordered | | NM_002789.4 | NP_002780.1 | |
| | | alpha type, 4 | ring-shaped 20S core structure. | | | | |
| | | | The core structure is composed | | | | |
| | | | of 4 rings of 28 non-identical | | | | |
| | | | subunits; 2 rings are composed | | | | |
| | | | of 7 alpha subunits and 2 rings | | | | |
| | | | are composed of 7 beta | | | | |
| | | | subunits. Proteasomes are | | | | |
| | | | distributed throughout | | | | |
| | | | eukaryotic cells at a high | | | | |
| | | | concentration and cleave | | | | |
| | | | peptides in an ATP/ubiquitin- | | | | |
| | | | dependent process in a non- | | | | |
| | | | lysosomal pathway. An | | | | |
| | | | essential function of a modified | | | | |
| | | | proteasome, the | | | | |
| | | | immunoproteasome, is the | | | | |
| | | | processing of class 1 MHC | | | | |
| | | | peptides. This gene encodes a | | | | |
| | | | member of the peptidase T1A | | | | |
| | | | family that is a 20S core alpha | | | | |
| | | | subunit. | | | | |
| RHOA | ARHA; ARH12; | ras homolog gene | Regulates a signal transduction | 3q21.3 | NM_001664.2 | NP_001655.1 | 387 |
| | RHO12; RHOH12 | family, member A | pathway linking plasma | | | | |
| | | | membrane receptors to the | | | | |
| | | | assembly of focal adhesions | | | | |
| | | | and actin stress fibers. May be | | | | |
| | | | an activator of PLCE1 | | | | |
| RPL28 | FLJ43307 | ribosomal protein | Ribosomes, the organelles that | 19q13.4 | NM_000991.3 | NP_000982.2 | 6158 |
| | | L28 | catalyze protein synthesis, | | | | |
| | | | consist of a small 40S subunit | | | | |
| | | | and a large 60S subunit. | | | | |
| | | | Together these subunits are | | | | |
| | | | composed of 4 RNA species | | | | |
| | | | and approximately 80 | | | | |
| | | | structurally distinct proteins. | | | | |
| | | | This gene encodes a ribosomal | | | | |
| | | | protein that is a component of | | | | |
| | | | the 60S subunit. The protein | | | | |
| | | | belongs to the L28E family of | | | | |
| | | | ribosomal proteins. | | | | |
| RPL3 | TARBP-B; | ribosomal protein | Ribosomes, the complexes that | 22q13 | NM_000967.3 | NP_000958.1 | 6122 |
| | MGC104284 | L3 | catalyze protein synthesis, | | NM_001033853.1 | NP_001029025.1 | |
| | | | consist of a small 40S subunit | | | | |
| | | | and a large 60S subunit. | | | | |
| | | | Together these subunits are | | | | |
| | | | composed of 4 RNA species | | | | |
| | | | and approximately 80 | | | | |
| | | | structurally distinct proteins. | | | | |
| | | | This gene encodes a ribosomal | | | | |
| | | | protein that is a component of | | | | |
| | | | the 60S subunit. The protein | | | | |
| | | | belongs to the L3P family of | | | | |
| | | | ribosomal proteins and it is | | | | |
| | | | located in the cytoplasm. | | | | |
| SFRS3 | SRp20 | splicing factor, | Potentially involved in RNA | 6p21 | NM_003017.3 | NP_003008.1 | 6428 |
| | | arginine/serine-rich 3 | processing in relation with | | | | |
| | | | cellular proliferation and/or | | | | |
| | | | maturation | | | | |
| SYNGR1 | MGC: 1939 | Synaptogyrin 1 | Encodes an integral membrane | 22q13.1 | NM_004711.4 | NP_004702.2 | 9145 |
| | | | protein associated with | | NM_145731.3 | NP_663783.1 | |
| | | | presynaptic vesicles in neuronal | | NM_145738.2 | NP_663791.1 | |
| | | | cells | | | | |
| TXNRD1 | TR; TR1; TXNR; | thioredoxin | This gene encods a member of | 12q23-q24.1 | NM_001093771.1 | NP_001087240.1 | 7296 |
| | TRXR1; GRIM-12; | reductase 1 | the family of pyridine | | NM_003330.2 | NP_003321.3 | |
| | MGC9145 | | nucleotide oxidoreductases. | | NM_182729.1 | NP_877393.1 | |
| | | | Reduced thioredoxins as well as | | NM_182742.1 | NP_877419.1 | |
| | | | other substrates, and plays a | | NM_182743.1 | NP_877420.1 | |
| | | | role in selenium metabolism | | | | |
| | | | and protection against oxidative stress. | | | | |

As used herein, the term “nucleic acid” refers to single or multiple stranded molecules which may be DNA or RNA, or any combination thereof, including modifications to those nucleic acids. The nucleic acid may represent a coding strand or its complement, or any combination thereof. Nucleic acids may be identical in sequence to the sequences which are naturally occurring for any of the moieties discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides), a reduction in the AT content of AT rich regions, or replacement of non-preferred codon usage of the expression system to preferred codon usage of the expression system. The nucleic acid can be directly cloned into an appropriate vector, or if desired, can be modified to facilitate the subsequent cloning steps. Such modification steps are routine, an example of which is the addition of oligonucleotide linkers which contain restriction sites to the termini of the nucleic acid. General methods are set forth in in Sambrook et al. (2001) Molecular Cloning—A Laboratory Manual (3rd ed.) Vol. 1-3, Cold Spring Harbor Laboratory, Cold Spring Harbor Press, NY, (Sambrook).

Once the nucleic acid sequence is obtained, the sequence encoding the specific amino acids can be modified or changed at any particular amino acid position by techniques well known in the art. For example, PCR primers can be designed which span the amino acid position or positions and which can substitute any amino acid for another amino acid. Alternatively, one skilled in the art can introduce specific mutations at any point in a particular nucleic acid sequence through techniques for point mutagenesis. General methods are set forth in Smith, M. “In vitro mutagenesis” Ann. Rev. Gen., 19:423-462 (1985) and Zoller, M. J. “New molecular biology methods for protein engineering” Curr. Opin. Struct. Biol., 1:605-610 (1991), which are incorporated herein in their entirety for the methods. These techniques can be used to alter the coding sequence without altering the amino acid sequence that is encoded.

The sequences contemplated herein include full-length wild-type (or native) sequences, as well as allelic variants, variants, fragments, homologs or fusion sequences that retain the ability to function as the cellular nucleic acid or protein involved in viral infection. In certain examples, a protein or nucleic acid sequence has at least 50% sequence identity, for example at least 50%, 55%, 60%, 65%, 70%, 75%, 80%, 85%, 90%, 93%, or 98% sequence identity to a native sequence set forth in Table 1. In other examples, a nucleic acid sequence involved in viral infection has a sequence that hybridizes to a sequence set forth in Table 1 and retains the activity of the sequence set forth in Table 1. For example, a nucleic acid that hybridizes to an AZIN1 nucleic acid sequence set forth in Table 1 (for example the nucleic acid sequence set forth under GenBank Accession No. NM_{—}015878.4 or NM_{—}148174.2) and encodes a protein that retains AZIN1 activity is contemplated by the present invention. Such sequences include the genomic sequence for the genes set forth in Table 1. The examples set forth above for AZIN1 are merely illustrative and should not be limited to AZIN1 as the analysis set forth in this example applies to every nucleic acid and protein listed in Table 1.

Unless otherwise specified, any reference to a nucleic acid molecule includes the reverse complement of the nucleic acid. Except where single-strandedness is required by the text herein (for example, a ssRNA molecule), any nucleic acid written to depict only a single strand encompasses both strands of a corresponding double-stranded nucleic acid. For example, depiction of a plus-strand of a dsDNA also encompasses the complementary minus-strand of that dsDNA. Additionally, reference to the nucleic acid molecule that encodes a specific protein, or a fragment thereof, encompasses both the sense strand and its reverse complement Fragments of the nucleic acids set forth in Table 1 and throughout the specification are also contemplated. These fragments can be utilized as primers and probes to amplify, inhibit, or detect any of the nucleic acids or genes set forth in Table 1.

Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning, second edition. Cold Spring Harbor Laboratory, Plainview, N.Y. (chapters 9 and 11). The following is an exemplary set of hybridization conditions and is not limiting:

Very High Stringency (Detects Sequences that Share 90% Identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Detects Sequences that Share 80% Identity or Greater)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Detects Sequences that Share Greater than 50% Identity)

Hybridization: 6×SSC at RT to 55° C. for 16-20 hours

Wash at least twice: 2×-3×SSC at RT to 55° C. for 20-30 minutes each.

Also provided is a vector, comprising a nucleic acid set forth herein. The vector can direct the in vivo or in vitro synthesis of any of the proteins or polypeptides described herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of toe inserted gene or hybrid gene (See generally, Sambrook et al.). The vector, for example, can be a plasmid. The vectors can contain genes conferring hygromycin resistance, ampicillin resistance, gentamicin resistance, neomycin resistance or other genes or phenotypes suitable for use as selectable markers, or methotrexate resistance for gene amplification.

There are numerous other E. coli (Escherichia coli) expression vectors, known to one of ordinary skill in the art, which are useful for the expression of the nucleic acid insert Other microbial hosts suitable for use include bacilli, such as Bacillus subtilis, and other enterobacteriaceae, such as Salmonella, Serratia, and various Pseudomonas species. In these prokaryotic hosts one can also make expression vectors, which will typically contain expression control sequences compatible with the host cell (e.g., an origin of replication). In addition, any number of a variety of well-known promoters will be present, such as the lactose promoter system, a tryptophan (Tip) promoter system, a beta-lactamase promoter system, or a promoter system from phage lambda. Additionally, yeast expression can be used. The invention provides a nucleic acid encoding a polypeptide of the present invention, wherein the nucleic acid can be expressed by a yeast cell. More specifically, the nucleic acid can be expressed by Pichia pastoris or S. cerevisiae.

Mammalian cells also permit the expression of proteins in an environment that favors important post-translational modifications such as folding and cysteine pairing, addition of complex carbohydrate structures, and secretion of active protein. Vectors useful for the expression of active proteins are known in the an and can contain genes conferring hygromycin resistance, genticin or G418 resistance, or other genes or phenotypes suitable for use as selectable markers, or methotrexate resistance for gene amplification. A number of suitable host cell lines capable of secreting intact human proteins have been developed in the an, and include the CHO cell lines, HeLa cells, COS-7 cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc.

The expression vectors described herein can also include nucleic acids of the present invention under the control of an inducible promoter such as the tetracycline inducible promoter or a glucocorticoid inducible promoter. The nucleic acids of the present invention can also be under the control of a tissue-specific promoter to promote expression of the nucleic acid in specific cells, tissues or organs. Any regulatable promoter, such as a metallothionein promoter, a heat-shock promoter, and other regulatable promoters, of which many examples are well known in the an are also contemplated. Furthermore, a Cre-loxP inducible system can also be used, as well as a Flp recombinase inducible promoter system, both of which are known in the art.

Insect cells also permit the expression of mammalian proteins. Recombinant proteins produced in insect cells with baculovirus vectors undergo post-translational modifications similar to that of wild-type proteins. The invention also provides for the vectors containing the contemplated nucleic acids in a host suitable for expressing the nucleic acids. The host cell can be a prokaryotic cell, including, for example, a bacterial cell. More particularly, the bacterial cell can be an E. coli cell. Alternatively, the cell can be a eukaryotic cell, including, for example, a Chinese hamster ovary (CHO) cell, a COS-7 cell, a HELA cell, an avian cell, a myeloma cell, a Pichia cell, or an insect cell. A number of other suitable host cell lines have been developed and include myeloma cell lines, fibroblast cell lines, a cell line suitable for infection by a pathogen, and a variety of tumor cell lines such as melanoma cell lines. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, Lipofectamine, or lipofectin mediated transfection, electroporation or any method now known or identified in the future can be used for other eukaryotic cellular hosts.

### Polypeptides

The present invention provides isolated polypeptides comprising the polypeptide or protein sequences set forth under the GenBank Accession Nos. set forth in Table 1. The present invention also provides fragments of these polypeptides. These fragments can be of sufficient length to serve as antigenic peptides for the generation of antibodies. The present invention also contemplates functional fragments that possess at least one activity of a gene or gene product listed in Table 1, for example, involved in viral infection. It will be known to one of skill in the an that each of the proteins set forth herein possess other properties, such as for example, enzymatic activity of AZTN1, ATPase activity of NAV3, transporter activity for Ost-alpha etc. Fragments and variants of the proteins set forth herein can include one or more conservative amino acid residues as compared to the amino acid sequence listed under their respective GenBank Accession Nos.

By “isolated polypeptide” or “purified polypeptide” is meant a polypeptide that is substantially free from the materials with which the polypeptide is normally associated in nature or in culture. The polypeptides of the invention can be obtained, for example, by extraction from a natural source if available (for example, a mammalian cell), by expression of a recombinant nucleic acid encoding the polypeptide (for example, in a cell or in a cell-free translation system), or by chemically synthesizing the polypeptide. In addition, a polypeptide can be obtained by cleaving full-length polypeptides. When the polypeptide is a fragment of a larger naturally occurring polypeptide, the isolated polypeptide is shorter than and excludes the full-length, naturally occurring polypeptide of which it is a fragment.

Also provided by the present invention is a polypeptide comprising an amino acid sequence at least about 50%, 55%, 60%, 65%, 70%, 75%, 80%, 85%, 90%, 95%, 96%, 97%, 98% or 99% identical to the polypeptide sequences set forth under the GenBank Accession Nos. disclosed herein.

It is understood that as discussed herein the use of the terms “homology” and “identity” mean the same thing as similarity. Thus, for example, if the use of the word homology is used to refer to two non-natural sequences, it is understood that this is not necessarily indicating an evolutionary relationship between these two sequences, but rather is looking al the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related.

In general, it is understood that one way to define any known variants and derivatives or those that might arise, of the disclosed nucleic acids and polypeptides herein, is through defining the variants and derivatives in terms of homology to specific known sequences. In general, variants of nucleic acids and polypeptides herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two polypeptides or nucleic acids. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needle man and Wunsch, J. Mol. Biol. 48:443 (1970), by the search for similarity method of Pearson and Lipman. Proc. Natl. Acad. Sci. U.S. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174:247-250 (1999) available from the National Center for Biotechnology Information (http://www.ncbi.nlm.nih.gov/blast/b12seq/b12.html)), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if (be first sequence is calculated to have 80 percent homology to the second sequence using each of calculation methods (although, in practice, the different calculation methods will often result in different calculated homology percentages).

Also provided by the present invention are polypeptides set forth under the GenBank Accession Nos. disclosed herein, or fragments thereof, with one or more conservative amino acid substitutions. These conservative substitutions are such that a naturally occurring amino acid is replaced by one having similar properties. Such conservative substitutions do not alter the function of the polypeptide. For example, conservative substitutions can be made according to the following table:

TABLE 1
Amino Acid Substitutions

| Original Residue | Exemplary Substitutions |
|---|---|
| Arg | Lys |
| Asn | Gln |
| Asp | Glu |
| Cys | Ser |
| Gln | Asn |
| Glu | Asp |
| Gly | Pro |
| His | Gln |
| Ile | leu; val |
| Leu | ile; val |
| Lys | arg; gln |
| Met | leu; ile |
| Phe | met; leu; tyr |
| Ser | Thr |
| Thr | Ser |
| Trp | Tyr |
| Tyr | trp; phe |
| Val | ile; leu |

Thus, it is understood that, where desired, modifications and changes may be made in the nucleic acid encoding the polypeptides of this invention and/or amino acid sequence of the polypeptides of the present invention and still obtain a polypeptide having like or otherwise desirable characteristics. Such changes may occur in natural isolates or may be synthetically introduced using site-specific mutagenesis, the procedures for which, such as mis-match polymerase chain reaction (PCR), are well known in the art. For example, certain amino acids may be substituted for other amino acids in a polypeptide without appreciable loss of functional activity. It is thus contemplated that various changes may be made in the amino acid sequence of the polypeptides of the present invention (or underlying nucleic acid sequence) without appreciable loss of biological utility or activity and possibly with an increase in such utility or activity. Thus, it is clear that naturally occurring variations in the polypeptide sequences set forth herein as well as genetically engineered variations in the polypeptide sequences set forth herein are contemplated by the present invention. By providing the genomic location of genes that are involved in viral infection, the present invention has also provided the genomic location of any variant sequences of these genes. Thus, based on the information provided herein, it would be routine for one of skill in the an to identify and sequence the genomic region identified by applicants and identify variant sequences of the genes set forth herein. It would also be routine for one of skill in the art to utilize comparison tools and bioinformatics techniques to identify sequences from other species that are homologs of the genes set forth herein and are also necessary for infection, but not necessary for survival of the cell.

### Methods of Decreasing Infection

The present invention provides a method of decreasing infection in a cell by a pathogen comprising decreasing expression or activity of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1.

As stated above, an infection can be a viral infection, bacterial infection, fungal infection or a parasitic infection, to name a few. A decrease or inhibition of infection can occur in a cell, in vitro, ex vivo or in vivo. As utilized throughout, the term “infection” encompasses all phases of pathogenic life cycles including, but not limited to, attachment to cellular receptors, entry, internalization, disassembly, replication, genomic integration of pathogenic sequences, transcription of viral RNA, translation of viral RNA, transcription of host cell mRNA, translation of host cell mRNA, proteolytic cleavage of pathogenic proteins or cellular proteins, assembly of particles, endocytosis, cell lysis, budding, and egress of the pathogen from the cells. Therefore, a decrease in infection can be a decrease in attachment to cellular receptors, a decrease in entry, a decrease in internalization, a decrease in disassembly, a decrease in replication, a decrease in genomic integration of pathogenic sequences, a decrease in translation of mRNA, a decrease in proteolytic cleavage of pathogenic proteins or cellular proteins, a decrease in assembly of particles, a decrease in endocytosis, a decrease in cell lysis, a decrease in budding, or a decrease in egress of the pathogen from the cells. This decrease does not have to be complete as this can range from a slight decrease to complete ablation of the infection. A decrease in infection can be at least about 10%, 20%, 30%, 40%, 30%, 60.70%, 80%, 90%, 93%, 100% or any other percentage decrease in between these percentages as compared to the level of infection in a cell wherein expression or activity of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 has not been decreased.

In the methods set forth herein, expression of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 can be inhibited, for example, by inhibiting transcription of the gene, or inhibiting translation of its gene product. Similarly, the activity of a gene product (for example, an mRNA, a polypeptide or a protein) can be inhibited, either directly or indirectly. Inhibition or a decrease in expression does not have to be complete as this can range from a slight decrease in expression to complete ablation of expression. For example, expression can be inhibited by about 10%, 20%, 30%, 40%, 30%, 60%, 70%, 80%, 90%, 93%, 99%, 100% or any percentage in between as compared to a control cell wherein the expression of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 has not been decreased or inhibited. Similarly, inhibition or decrease in the activity of a gene product does not have to be complete as this can range from a slight decrease to complete ablation of the activity of the gene product. For example, the activity of a gene product can be inhibited by about 10%, 20%, 30%, 40%, 50%, 60%, 70%, 80%, 90%, 93%, 99%, 100% or any percentage in between as compared to a control cell wherein activity of a AZIN1, CENPL, C6orf111 (SFRS18), INHBA, NAV3, ODZ2, Ost-alpha, Ost-beta, PSMA4, RHOA, RPL28, RPL3, SFRS3, SYNCR1 or TXNRD1 gene product has not been decreased or inhibited. As utilized herein, “activity of a gene product” can be an activity that is involved in pathogenicity, for example, interacting directly or indirectly, with pathogen, e.g. viral protein or viral nucleic acids, or an activity that the gene product performs in a normal cell, i.e. in a non-infected cell. Depending on the gene product, one of skill in the art would know bow to assay for an activity that is involved in pathogenicity, an activity that is involved in normal cellular function, or both. As set forth above, an activity of the proteins and nucleic acids listed herein can be the ability to bind or interact with other proteins. Therefore, the present invention also provides a method of decreasing infection by inhibiting or decreasing the interaction between any of the proteins of the present invention and other cellular proteins, such as, for example, receptors, enzymes, nucleic acids and hormones, provided that such inhibition correlates with decreasing infection by the pathogen. Also provided is a method of decreasing infection by inhibiting or decreasing the interaction between any of the proteins of the present invention and a viral, bacterial, parasitic or fungal protein (i.e. a non-host protein).

The cells of the present invention can be prokaryotic or eukaryotic, such as a cell from an insect, fish, crustacean, mammal, bird, reptile yeast or a bacterium, such as E. coli. The cell can be part of an organism, or pan of a cell culture, such as a culture of mammalian cells or a bacterial culture. Therefore, the cell can also be pan of a population of cells. The cell(s) can also be in a subject.

Examples of viral infections include but are not limited to, infections caused by RNA viruses (including negative stranded RNA viruses, positive stranded RNA viruses, double stranded RNA viruses and retroviruses) and DNA viruses.

Examples of RNA viruses include, but are not limited to picornaviruses, which include aphthoviruses (for example, foot and mouth disease virus O, A, C, Asia 1. SAT1, SAT2 and SAT3), cardioviruses (for example, encephalomycarditis virus and Theiller's murine encephalomyelitis virus), enteroviruses (for example polioviruses 1, 2 and 3, human enteroviruses A-D, bovine enteroviruses 1 and 2, human coxsackieviruses A1-A22 and A24, human coxsackieviruses B1-B5, human echoviruses 1-7, 9, 11-12, 24, 27, 29-33, human enteroviruses 68-71, porcine enteroviruses 8-10 and simian enteroviruses 1-18), erboviruses (for example, equine rhinitis virus), hepatovirus (for example human hepatitis A virus and simian hepatitis A virus), kobuviruses (for example, bovine kobuvirus and Aichi virus), parechoviruses (for example, human parechovirus 1 and human parechovirus 2), rhino virus (for example, human rhinovirus 1-100 and bovine rhinoviruses 1-3) and teschoviruses (for example, porcine teschovirus).

Additional examples of RNA viruses include caliciviruses, which include noroviruses (for example, Norwalk virus), sapoviruses (for example, Sapporo virus), lagoviruses (for example, rabbit hemorrhagic disease virus and European brown hare syndrome) and vesiviruses (for example vesicular exanthema of swine virus and feline calicivirus).

Other RNA viruses include astroviruses, which include mamastorviruses and avastroviruses. Togaviruses are also RNA viruses. Togaviruses include alphaviruses (for example, Sindbis virus, Chikungunya virus, Semliki Forest virus. Western equine encephalitis, Getah virus, Everglades virus, Venezuelan equine encephalitis virus and Aura virus) and rubella viruses. Additional examples of RNA viruses include the flaviviruses (for example, tick-borne encephalitis virus, Tyuleniy virus, Aroa virus, Dengue virus (types 1 to 4), Kedougou virus, Japanese encephalitis virus (JEV), West Nile virus (WNV), Kokobera virus, Ntaya virus, Spondweni virus, Yellow fever virus, Entebbe bat virus, Modoc virus, Rio Bravo virus. Cell fusing agent virus, pestivirus, GB virus A, GBV-A like viruses, GB virus C Hepatitis G virus, hepacivirus (hepatitis C virus (HCV)) all six genotypes), bovine viral diarrhea virus, and GB virus B).

Other examples of RNA viruses are the coronaviruses which include, human respiratory coronaviruses such as SARS-CoV, HCoV-229E, HCoV-NL63 and HCoV-OC43. Coronaviruses also include bat SARS-like CoV, turkey coronavirus, chicken coronavirus, feline coronavirus and canine coronavirus. Additional RNA viruses include arteriviruses (for example, equine arterivirus, porcine reproductive and respiratory syndrome virus, lactate dehydrogenase elevating virus of mice and simian hemorraghic fever virus). Other RNA viruses include the rhabdoviruses, which include lyssaviruses (for example, rabies, Lagos bat virus, Mokola virus, Duvenhage virus and European bat lyssavirus), vesiculoviruses (for example, VSV-Indiana, VSV-New Jersey, VSV-Alagoas, Pity virus, Cecal virus, Maraba virus, Isfahan virus and Chandipura virus), and ephemeroviruses (for example, bovine ephemeral fever virus, Adelaide River virus and Berrimah virus). Additional example of RNA viruses include the filoviruses. These include the Marburg and Ebola viruses (for example, EBOV-Z, EBOV-S, EBOV-IC and EBOV-R.

The paramyxoviruses are also RNA viruses. Examples of these viruses are the rubula viruses (for example, mumps, parainfluenza virus 5, human parainfluenza virus type 2, Mapuera virus and porcine rubulavirus), avula viruses (for example, Newcastle disease virus), respoviruses (for example, Sendai virus, human parainfluenza virus type 1 and type 3, bovine parainfluenza virus type 3), henipaviruses (for example, Hendra virus and Nipah virus), morbilloviruses (for example, measles. Cetacean morvilliirus, Canine distemper virus, Peste-des-petits-ruminants virus, Phocine distemper virus and Rinderpest virus), pneumoviruses (for example, human respiratory syncytial virus A2, B1 and S2, bovine respiratory syncytial virus and pneumonia virus of mice), metapneumoviruses (for example, human metapneumovirus and avian metapneumovirus). Additional paramyxoviruses include Fer-de-Lance virus, Tupaia paramyxovirus, Menangle virus, Tioman virus, Beilong virus, J virus, Moss man virus, Salem virus and Nariva virus.

Additional RNA viruses include the orthomyxoviruses. These viruses include influenza viruses (e.g., influenza A, B and C viruses, as well as avian influenza (for example, strains H5N1, H5N2, H7N1, H7N7 and H9N2)) thogotoviruses and isaviruses. Orthobunyaviruses (for example, Akabane virus, California encephalitis, Cache Valley virus, Snowshoe hare virus.) neuroviruses (for example, Nairobi sheep virus, Crimean-Congo hemorrhagic fever virus Group and Hughes virus), phleboviruses (for example, Candiru, Punta Toro, Rift Valley Fever, Sandfly Fever, Naples, Toscana, Sicilian and Chagres), and hantaviruses (for example, Hantaan, Dobrava, Seoul, Puumala, Sin Nombre, Bayou, Black Creek Canal, Andes and Thottapalayam) are also RNA viruses. Arenaviruses such as lymphocytic choriomeningitis virus, Lassa fever virus, Argentine hemorrhagic fever virus, Bolivian hemorrhagic fever virus, Venezuelan hemorrhagic fever virus, SABV and WWAV are also RNA viruses. Boma disease virus is also an RNA virus. Hepatitis D (Delta) virus and hepatitis E are also RNA viruses.

Additional RNA viruses include reoviruses, rotaviruses, bimaviruses, chrysoviruses, cystoviruses, hypoviroses, partitiviruses and totoviruses. Orbiviruses such as African horse sickness virus. Blue tongue virus, Changuinola virus, Chenuda virus, Chobar Gorge Corripana virus, epizootic hemorraghic disease virus, equine encephalosis virus, Eubenangee virus, Ieri virus. Great Island virus, Lebombo virus, Orungo virus, Palyam virus, Peruvian Horse Sickness virus, St. Croix River virus, Umatilla virus. Wad Medani virus, Wallal virus, Warrego virus and Wongorr virus are also RNA viruses.

Retroviruses include alpharetroviruses (for example, Rous sarcoma virus and avian leukemia virus), belaretroviruses (for example, mouse mammary tumor virus, Mason-Pfizer monkey virus and Jaagsiekte sheep retrovirus), gammaretroviruses (for example, murine leukemia virus and feline leukemia virus, deltraretroviruses (for example, human T cell leukemia viruses (HTLV-1, HTLV-2), bovine leukemia virus, STLV-1 and STLV-2), epsilonretroviruses (for example. Walleye dermal sarcoma virus and Walleye epidermal hyperplasia virus 1), reticuloendotheliosis virus (for example, chicken syncytial virus, lentiviruses (for example, human immunodeficiency virus (HIV) type 1, human immunodeficiency virus (HIV) type 2, simian immunodeficiency virus, equine infectious anemia virus, feline immunodeficiency virus, caprine arthritis encephalitis virus and Visna maedi virus) and spumaviruses (for example, human foamy virus and feline syncytia-forming virus).

Examples of DNA viruses include polyoma viruses (for example, simian virus 40, simian agent 12, BK virus, JC virus, Merkel Cell polyoma virus, bovine polyoma virus and lymphotrophic papovavirus), papillomaviruses (for example, human papillomavirus, bovine papillomavirus, adenoviruses (for example, adenoviruses A-F, canine adenovirus type 1, canined adeovirus type 2), circoviruses (for example, porcine circovirus and beak and feather disease virus (BFDV)), parvoviruses (for example, canine parvovirus), erythroviruses (for example, adeno-associated virus types 1-8), betaparvoviruses, amdoviruses, densoviruses, iteraviruses, brevidensoviruses, pefudensoviruses, herpes viruses (for example, herpes simplex virus 1, herpes simplex virus 2, varicella-zoster virus, Epstein-Barr virus, cytomegalovirus, Kaposi's sarcoma associated herpes virus, human herpes virus-6 variant A, human herpes virus-6 variant B and cercophithecine herpes virus 1 (B virus)), poxviruses (for example, smallpox (variola), cowpox, monkeypox, vaccinia. Uasin Gishu, camelpox, psuedocowpox, pigeonpox, horsepox, fowlpox, turkeypox and swinepox), and hepadnaviruses (for example, hepatitis B and hepatitis B-like viruses). Chimeric viruses comprising portions of more than one viral genome are also contemplated herein.

For animals, in addition to the animal viruses listed above, viruses include, but are not limited to, the animal counterpart to any above listed human virus. The provided genes can also decrease infection by newly discovered or emerging viruses. Such viruses are continuously updated on http://en.wikepedia.org/wiki/Virus and www.virology.net.

Examples of bacterial infections include, but are not limited to infections caused by the following bacteria: Listeria (sp.), Franciscella tularensis, Mycobacterium tuberculosis, Rickettsia (all types), Ehrlichia, Chylamida. Further examples of bacteria that can be targeted by the present methods include M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Kingella kingae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Examples of parasitic infections include, but are not limited to infections caused by the following parasites: Cryptosporidium, Plasmodium (all species), American trypanosomes (T. cruzi). African trypanosomes, Acanthamoeba, Entaoeba histolytica, Angiostrongylus, Anisakis, Ascaris, Babesia, Balantidium, Baylisascaris, lice, ticks, mites, fleas, Capillaria, Clonorchis, Chilomastix mesnili, Cyclospora, Diphyllobothrium, Dipylidium caninum, Fasciola, Giardia, Gnathostoma, Hetetophyes, Hymnolepsis, Isospora, Loa loa, Microsporidia, Naegleria, Toxocara, Onchocerca, Opisthorchis, Paragonimus, Baylisascaris, Strongyloides, Taenia, Trichomonas and Trichuris.

Furthermore, examples of protozoan and fungal species contemplated within the present methods include, but are not limited to, Plasmodium falciparum, other Plasmodium species, Toxoplasma gondii, Pneumocystis carinii, Trypanosoma cruzi, other trypanosomal species, Leishmania donovani, other Leishmania species, Theiteria annulata, other Theileria species, Eimeria tenella, other Eimeria species, Histoplasma capsulatum, Cryptococcus neoformans, Blastomyces dermatitidis, Coccidioides immitis, Paracoccidioides brasiliensis, Penicillium mameffei, and Candida species. The provided genes can also decrease infection by newly discovered or emerging bacteria, parasites or fungi, including multidrug resistant strains of same.

The present invention also provides method of decreasing infection in a cell by a pathogen comprising administering anisomycin or an anisomycin derivative to the cell. The cell can be in vitro, ex vivo or in vivo. The structure of anisomycin is set forth below. Anisomycin is also known as flagecidin or as [(2S,3R,4R)-4-hydroxy-2-[(4-methoxyphenyl)methyl]pyrrolidin-3-yl]acetate. Any pharmaceutically acceptable salt, ester, amides or prodrug of anisomycin or of an anisomycin derivative can also be used. Anisomycin derivatives include but are not limited to: 3-O-carbamoyldeacetylanisomycin, 3-O-methylcarbamoyldeacetylanisomycin, 3-O-ethylcarbamoyldeacetylanisomycin, 3-O-propylcarbamoyldeacetylanisomycin, 3-O-(3-phenylpropyl)carbamoyldeacetylanisomycin, 3-O-cyclopropylcarbamoyldeacetylanisomycin, 3-O-dimethylcarbamoyldeacetylanisomycin, 3-O-(2-hydroxyethyl)carbamoyldeacetylanisomycin, 3-O-(2-dimethylaminoethyl)carbamoyldeacetylanisomycin, 3-O-(3-dimethylaminopropyl)carbamoyldeacetylanisomycin, 3-O-phenylcarbamoyldeacetylanisomycin, 4-O-acetyl-3-O-methylcarbamoyldeacetylanisomycin, 4-O-heptanoyl-3-O-methylcarbamoyldeacetylanisomycin, 4-O-octadecanoyl-3-O-methylcarbamoyldeacetylanisomycin, 4-O-acetyl-3-O-carbamoyldeacetylanisomycin, 4-O-hexanoyl-3-O-carbamoyldeacetylanisomycin, 4-O-heptanoyl-3-O-carbamoyldeacetylanisomycin, 4-O-dodecanoyl-3-O-carbamoyldeacetylanisomycin, 4-O-octadecanoyl-3-O-carbamoyldeacetylanisomycin, 3-O-methyldeacetylanisomycin, 3-O-ethyl-deacetylanisomycin, 3-O-methoxymethyldeacetylanisomycin, 3-O-(2-methoxyethoxy)methyldeacetylanisomycin and other anisomycin derivatives set forth in U.S. Pat. No. 5,463,080 hereby incorporated in its entirely by this reference.

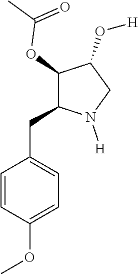 [SMILES: COc1ccc(C[C@@H]2NC[C@@H](O)[C@@H]2OC(C)=O)cc1]

The present invention also provides a method of decreasing the toxicity of a toxin in a cell comprising decreasing expression or activity of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1. The cell can be in vitro, ex vivo or in vivo. Toxins can include, but are not limited to, a bacterial toxin, neurotoxins, such as botulinum neurotoxins, mycotoxins, ricin, Clostridium perfringens toxins, saxitoxins, tetrodotoxins, abrin, conotoxins, Staphylococcal toxins, E. coli toxins, streptococcal toxins, shigatoxins, T-2 toxins, anthrax toxins, chimeric forms of the toxins listed herein, and the like. The decrease in toxicity can be at least about 10%, 20%, 30%, 40%, 50%, 60%, 70%, 80%, 90%, 95%, 100% or any other percentage decrease in between these percentages as compared to the level of toxicity in a cell wherein expression or activity of AZIN1. CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 has not been decreased.

Toxicity can be measured, for example, via a cell viability, apopotosis assay, LDH release assay or cytotoxicity assay (See, for example, Kehl-Fie and St. Geme “Identification and characterization of an RTX toxin in the emerging pathogen Kingella kingae,” J. Bacteriol. 189(2):430-6 (2006) and Kirby “Anthrax Lethal Toxin Induces Human Endothelial cell Apoptosis,” Infection and Immunity 72:430-439 (2004), both of which are incorporated herein in their entireties by this reference.)

In the methods of the present invention, contacting the cell with any composition that can decrease expression or activity of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 provides a decrease in the effective gene product, albeit RNA or protein. For example, the composition can comprise a chemical, a small or large molecule (organic or inorganic), a drug, a protein, a peptide, a cDNA, an antibody, a morpholino, a triple helix molecule, an aptamer, an siRNA, a shRNA, an miRNA, an antisense RNA, a ribozyme or any other compound now known or identified in the future that decreases the expression and/or activity of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1. A decrease in expression or activity can occur by decreasing transcription of mRNA or decreasing translation of RNA. A composition can also be a mixture or “cocktail” of two or more of the compositions described herein.

These compositions can be used alone or in combination with other therapeutic agents such as antiviral compounds, antibacterial agents, antifungal agents, antiparasitic agents, anti-inflammatory agents, anti-cancer agents, etc. All of the compounds described herein can be contacted with a cell in vitro, ex vivo or in vivo.

Examples of antiviral compounds include, but are not limited to, amantadine, rimantadine, zanamavir and oseltamavir (Tamiflu) for the treatment of flu and its associated symptoms. Antiviral compounds useful in the treatment of HIV include Combivir® (lamivudine-zidovudine), Crixivan® (indinavir), Emtriva® (emtricitabine), Epivir® (lamivudine), Fortovase® (saquinavir-sg), Hivid® (zalcitabine), Invirase® (saquinavir-hg), Kaletra® (lopinavir-ritonavir), Lexiva™ (fosamprenavir), Norvir® (ritonavir), Retrovir® (zidovudine) Sustiva® (efavirenz), Videx EC® (didanosine), Videx® (didanosine), Viracept® (nelfinavir) Viramune® (nevirapine), Zerit® (stavudine), Ziagen® (abacavir), Fuzeon® (enfuvirtide) Rescriptor® (delavirdine), Reyataz® (atazanavir), Trizivir® (abacavir-lamivudine-zidovudine) Viread® (tenofovir disoproxil fumarate) and Agenerase® (amprenavir). Other antiviral compounds useful in the treatment of Ebola and other filoviruses include ribavirin and cyanovirin-N(CV-N). For the treatment of herpes virus, Zovirax® (acyclovir) is available. Antibacterial agents include, but are not limited to, antibiotics (for example, penicillin and ampicillin), sulfa Drugs and folic acid Analogs, Beta-Lactams, aminoglycosides, tetracyclines, macrolides, lincosamides, streptogramins, fluoroquinolones, rifampin, mupirocin, cycloserine, aminocyclitol and oxazolidinones.

Antifungal agents include, but are not limited to, amphotericin, nystatin, terbinafine, itraconazole, fluconazole, ketoconazole, and griselfulvin.

Antiparasitic agents include, but are not limited to, anthelmintics, antinematodal agents, antiplatyhelmintic agents, antiprotozoal agents, amebicides, antimalarials, antitrichomonal agents, aoccidiostats and trypanocidal agents.

### Antibodies

The present invention also provides antibodies that specifically bind to the gene products, proteins and fragments thereof set forth in Table 1. The antibody of the present invention can be a polyclonal antibody or a monoclonal antibody. The antibody of the invention selectively binds a polypeptide. By “selectively binds” or “specifically binds” is meant an antibody binding reaction which is determinative of the presence of the antigen (in the present case, a polypeptide set forth in Table 1 or antigenic fragment thereof among a heterogeneous population of proteins and other biologies). Thus, under designated immunoassay conditions, the specified antibodies bind preferentially to a particular peptide and do not bind in a significant amount to other proteins in the sample. Preferably, selective binding includes binding at about or above 1.5 times assay background and the absence of significant binding is less than 1.5 times assay background.

This invention also contemplates antibodies that compete for binding to natural interactors or ligands to the proteins set forth in Table 1. In other words, the present invention provides antibodies that disrupt interactions between the proteins set forth in Table 1 and their binding partners. For example, an antibody of the present invention can compete with a protein for a binding site (e.g. a receptor) on a cell or the antibody can compete with a protein for binding to another protein or biological molecule, such as a nucleic acid that is under the transcriptional control of a transcription factor set forth in Table 1. An antibody can also disrupt the interaction between a protein set forth in Table 1 and a pathogen, or the product of a pathogen. For example, an antibody can disrupt the interaction between a protein set forth in Table 1 and a viral protein, a bacterial protein, a parasitic protein, a fungal protein or a toxin. The antibody optionally can have either an antagonistic or agonistic function as compared to the antigen. Antibodies that antagonize pathogenic infection are utilized to decrease infection.

Preferably, the antibody binds a polypeptide in vitro, ex vivo or in vivo. Optionally, the antibody of the invention is labeled with a detectable moiety. For example, the detectable moiety can be selected from the group consisting of a fluorescent moiety, an enzyme-linked moiety, a biotin moiety and a radiolabeled moiety. The antibody can be used in techniques or procedures such as diagnostics, screening, or imaging. Anti-idiotypic antibodies and affinity-matured antibodies are also considered to be part of the invention.

As used herein, the term “antibody” encompasses chimeric antibodies and hybrid antibodies, with dual or multiple antigen or epitope specificities, and fragments, such as F(ab′)2, Fab′. Fab and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York. (1988)).

Also included within the meaning of “antibody” are conjugates of antibody fragments and antigen-binding proteins (single chain antibodies) as described, for example, in U.S. Pat. No. 4,704,692, the contents of which are hereby incorporated by reference.

Optionally, the antibodies are generated in other species and “humanized” for administration in humans. In one embodiment of the invention, the “humanized” antibody is a human version of the antibody produced by a germ line mutant animal. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat or rabbit having the desired specificity, affinity and capacity. In one embodiment, the present invention provides a humanized version of an antibody, comprising at least one, two, three, four, or up to all CDRs of a monoclonal antibody that specifically binds to a protein or fragment thereof set forth in Table 1. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of or at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al. Nature, 321:522-525 (1986); Riechmann et al. Nature, 332:323-327 (1988); and Presta. Curr. Op. Struct Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al. Nature, 321:522-525 (1986); Riechmann et al. Nature, 332:323-327 (1988); Verhoeyen et al. Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

### Peptides

Peptides that inhibit AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 expression or activity are also provided herein. Peptide libraries can be screened utilizing the screening methods set forth herein to identify peptides that inhibit activity of any of the genes or gene products set forth in Table 1. These peptides can be derived from a protein that binds to any of the genes or gene products set forth in Table 1. These peptides can be any peptide in a purified or non-purified form, such as peptides made of D- and/or L-configuration amino acids (in, for example, the form of random peptide libraries; see Lam et al. Nature 354:82-4, 1991), phosphopeptides (such as in the form of random or partially degenerate, directed phosphopeptide libraries; see, for example, Songyang et al. Cell 72:767-78, 1993).

siRNAs

Short interfering RNAs (siRNAs), also known as small interfering RNAs, are double-stranded RNAs that can induce sequence-specific post-transcriptional gene silencing, thereby decreasing gene expression (See, for example, U.S. Pat. Nos. 6,506,559, 7,056,704, 7,078,196, 6,107,094, 5,898,221, 6,573,099, and European Patent No. 1,144,623, all of which are hereby incorporated in their entireties by this reference). siRNas can be of various lengths as long as they maintain their function. In some examples, siRNA molecules are about 19-23 nucleotides in length, such as at least 21 nucleotides, for example at least 23 nucleotides. In one example, siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends. The direction of dsRNA processing determines whether the produced siRNA endonuclcase complex can cleave a sense or an antisense target RNA. Thus, siRNAs can be used to modulate transcription or translation, for example, by decreasing gene expression of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1. The effects of siRNAs have been demonstrated in cells from a variety of organisms, including Drosophila, C. elegans, insects, frogs, plants, fungi, mice and humans (for example. WO 02/44321; Gitlin et al. Nature 418:430-4, 2002; Caplen et al., Proc. Natl. Acad. Sci. 98:9742-9747, 2001; and Elbashir et al. Nature 411:494-B. 2001).

Utilizing sequence analysis tools, one of skill in the art can design siRNAs to specifically target AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 for decreased gene expression. siRNAs that inhibit or silence gene expression can be obtained from numerous commercial entities that synthesize siRNAs, for example, Ambion Inc. (2130 Woodward Austin, Tex. 78744-1832, USA). Qiagen Inc. (27220 Tumberry Lane, Valencia, Calif. USA) and Dharmacon Inc. (630 Crescent Drive, #100 Lafayette, Colo. 80026, USA). The siRNAs synthesized by Ambion Inc., Qiagen Inc. or Dharmacon Inc. can be readily obtained from these and other entities by providing a GenBank Accession No. for the mRNA of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1. In addition, siRNAs can be generated by utilizing Invitrogen's BLOCK-IT™ RNAi Designer https://rnaidesigner.invitrogen.com/rnaiexpress.

Examples of siRNA sequences that can be utilized in the methods described herein include, but are not limited, to those set forth below. Specifically, the sense siRNA sequences set forth below and sequences complementary to these sequences can be used alone or in combination with other sequences to inhibit gene expression. Also contemplated are siRNA sequences that are shorter or longer than the sequences set forth below. For example, an siRNA sequence comprising any of the sequences set forth below can be readily generated by adding nucleotides, on one or both ends of the siRNA, that flank these sequences in the full-length mRNA for the gene of interest Nucleotides can also be removed, from one or both ends of the siRNA to generate shorter siRNA sequences that retain their function. These sequences can comprise a 3′TT overhang and/or additional sequences that allow efficient cloning and expression of the si RNA sequences. All of the sequences disclosed herein can be cloned into vectors and utilized in vitro, ex vivo or in vivo to decrease gene expression. These si RNA sequences are merely exemplary as one of skill in the art would know that it is routine to utilize publicly available algorithms for the design of siRNA to target mRNA sequences. These sequences can then be assayed for inhibition of gene expression in vitro, ex vivo or in vivo.

### AZIN1

GGAACCGGAUUUGCUUGUU
CCAAGGUCUUACUACAUAU
GGAAUGUGCUAAGGAACUU
GCUGGAGAAAUUGGCUUUA
GGAAGAGGUUAAUCAUGUU
GGAACCGGAUUUGCUUGUU
CCAAGGUCUUACUACAUAU
GGAAUGUGCUAAGGAACUU
GCUGGAGAAAUUGGCUUUA

### CENPL

GCACCAGAGUCAACUCCUA
GGUUGCAUUCCUUCUGCAU
GCAUAAACAGUGGACUUUA
GCUGGUUCUGCUGUGUAUU
GGAGACUGUUUCAGAAGAU

### SFRS18

GCCCUUGAACCAGCAACAA
CCAAACAAUCAUGGGAAUU
CCAGAACAAUCACAACUUU
GCCAUCAUCAUUCAGGGAU
GCGUUCACCUAUUGCACUU

### INHBA

GCUUUGGCUGAGAGGAUUU
GGCUGAGAGGAUUUCUGUU
GGAUUUCUGUUGGCAAGUU
GGAGAUAGAGGAUGACAUU
GGCAGAAAUGAAUGAACUU

### NAV3

GCAGAAAUCAUCCAGAUUA
GCAAGACAACAGCAGCUAA
GCAGCCCUCUCUUCAAUAA
GCAUGCAGCUUGACAGAAA
GCCACAAGCCAUUCCAGUA

### ODZ2

GCAGAUGGGCACACCUUUA
GCAUCUGGCCUUCUACAAU
GCAUCUUUCCCUCUCGAAA
GCAACAACCCAGCACACAA
GCUGUCAAUCCCAUGGAUA

### QST-Alpha

GGAGGUGCUGAAGACCAAU
CCGUCUACCUGUACAAGAA
CCUGUACAAGAACACCCUU
GGUCAUGGUGGAAGGCUUU
CCUCCAUCUUCUCAGUCUU

### OST-Beta

GCUGCUGGAAGAGAUGCUU
GCAUCUCCCUGGAAUCAUU
CCUGGAAUCAUUCCAUCCU
GCAGCUGUGGUGGUCAUUA
GCUGUGGUGGUCAUUAUAA

### PSMA4

GGCAGGCAUAACUUCUGAU
GCAUAACUUCUGAUGCUAA
GCUCAUUGCUCAAAGGUAU
CCUUGUGAGCAGUUGGUUA
GGGAUAAGCACUAUGGCUU
GGCAGGCAUAACUUCUGAU
GCAUAACUUCUGAUGCUAA
GCUCAUUGCUCAAAGGUAU
CCUUGUGAGCAGUUGGUUA
GGGAUAAGCACUAUGGCUU
GGCAGGCAUAACUUCUGAU
GCAUAACUUCUGAUGCUAA
GCUCAUUGCUCAAAGGUAU
CCUUGUGAGCAGUUGGUUA
GGGAUAAGCACUAUGGCUU

### RHOA

CCGGAAGAAACUGGUGAUU
GGAGCCUGUGGAAAGACAU
CCUGUGGAAAGACAUGCUU
CCAGUUCCCAGAGGUGUAU
GGCAGAUAUCGAGGUGGAU

### RPL28

GCUCCAGUUUCCUGAUCAA
GCACUGAGCCCAAUAACUU
GCAAUUCCUUCCGCUACAA
CCGCUACAACGGACUGAUU
GCUACAACGGACUGAUUCA

### RPL3

GGACCUUCAAGACUGUCUU
GCAAGAGGCGUUUCUAUAA
GCCAAGUCAUCCGUGUCAU
CCAAGUCAUCCGUGUCAUU
GCAGGUACCUGUGAACCAA
GGACCUUCAAGACUGUCUU
GCAAGAGGCGUUUCUAUAA
GCAGGUACCUGUGAACCAA
CCAAGGGCAAAGGCUACAA
GCCAGGGCUACCUUAUCAA

### SFRS3

CCUGUCCAUUGGACUGUAA
UCCAUUGGACUGUAAGGUU
CCAUUGGACUGUAAGGUUU
GGACUGUAAGGUUUAUGUA
GGUUUAUGUAGGCAAUCUU

### SYNGR1

CGUCGUGUCUUGGCUGUUCUCCAUA
CGUGUCUUGGCUGUUCUCCAUAGUG
UAGUGGUGUUCGGCUCCAUCGUGAA
GAGGAGUUCUGCAUCUACAACCGCA
AGGAGUUCUGCAUCUACAACCGCAA
GCUUUCCUCUGGUUCGUGGGAUUCU

### TXNRD1

GCCAUGGUCCAACCUUGAA
GGUCCAACCUUGAAGGCUU
GCAUCAAGCAGCUUUGUUA
GCAAGACUCUCGAAAUUAU
GGAGCAUCCUAUGUCGCUU
GCCAUGGUCCAACCUUGAA
GGUCCAACCUUGAAGGCUU
GCAUCAAGCAGCUUUGUUA
GCAAGACUCUCGAAAUUAU
GGAGCAUCCUAUGUCGCUU
GCAUCAAGCAGCUUUGUUA
GCAAGACUCUCGAAAUUAU
GGAGCAUCCUAUGUCGCUU
CCAUUCUUCUUAGAGGAUU
GGAAAUCAUUGAAGGAGAA
GCAUCAAGCAGCUUUGUUA
GCAAGACUCUCGAAAUUAU
GGAGCAUCCUAUGUCGCUU
CCAUUCUUCUUAGAGGAUU
GGAAAUCAUUGAAGGAGAA
GCAUCAAGCAGCUUUGUUA
GCAAGACUCUCGAAAUUAU
GGAGCAUCCUAUGUCGCUU
CCAUUCUUCUUAGAGGAUU

shRNA

shRNA (short hairpin RNA) is a DNA molecule that can be cloned into expression vectors to express siRNA (typically 19-29 nt RNA duplex) for RNAi interference studies. shRNA has die following structural features: a short nucleotide sequence ranging from about 19-29 nucleotides derived from the target gene, followed by a short spacer of about 4-15 nucleotides (i.e. loop) and about a 19-29 nucleotide sequence that is the reverse complement of the initial target sequence.

### Antisense Nucleic Acids

Generally, the term “antisense” refers to a nucleic acid molecule capable of hybridizing to a portion of an RNA sequence (such as mRNA) by virtue of some sequence complementarity. The antisense nucleic acids disclosed herein can be oligonucleotides that are double-stranded or single-stranded, RNA or DNA or a modification or derivative thereof, which can be directly administered to a cell (for example by administering the antisense molecule to the subject), or which can be produced intracellularly by transcription of exogenous, introduced sequences (for example by administering to the subject a vector that includes the antisense molecule under control of a promoter).

Antisense nucleic acids are polynucleotides, for example nucleic acid molecules that are at least 6 nucleotides in length, at least 10 nucleotides, at least 15 nucleotides, at least 20 nucleotides, at least 100 nucleotides, at least 200 nucleotides, such as 6 to 100 nucleotides. However, antisense molecules can be much longer. In particular examples, the nucleotide is modified at one or more base moiety, sugar moiety, or phosphate backbone (or combinations thereof), and can include other appending groups such as peptides, or agents facilitating transport across the cell membrane (Letsinger et al., Proc. Natl. Acad. Sci. USA 1989, 86:6333-6; Lemaitre et al., Proc. Natl. Acad. Sci. USA 1987, 84:648-52; WO 88/09810) or blood-brain barrier (WO 89/10134), hybridization triggered cleavage agents (Krol et al., BioTechniques 1988, 6:958-76) or intercalating agents (Zon, Pharm. Res. 5:539-49, 1988). Additional modifications include those set forth in U.S. Pat. Nos. 7,176,296; 7,329,648; 7,262,489, 7,113,379; and 7,105,495.

Examples of modified base moieties include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid. 5-methyl-2-thiouracil, 3-(3-amino-3-N2-carboxypropyl) uracil, and 2,6-diaminopurine.

Examples of modified sugar moieties include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

In a particular example, an antisense molecule is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 15:6625-41, 1987). The oligonucleotide can be conjugated to another molecule, such as a peptide, hybridization triggered cross-linking agent, transport agent, or hybridization-triggered cleavage agent. Oligonucleotides can include a targeting moiety that enhances uptake of the molecule by host cells. The targeting moiety can be a specific binding molecule, such as an antibody or fragment thereof that recognizes a molecule present on the surface of the host cell.

In a specific example, antisense molecules that recognize a nucleic acid set forth herein, include a catalytic RNA or a ribozyme (for example see WO 90711364; WO 95/06764; and Sarver et al., Science 247:1222-5, 1990). Conjugates of antisense with a metal complex, such as terpyridylCu (II), capable of mediating mRNA hydrolysis, are described in Bashkin et al. (Appl. Biochem Biotechnol. 54:43-56, 1995). In one example, the antisense nucleotide is a 2′-0-methylribonucleotide (Inoue et al., Nucl. Acids Res. 15:6131-48, 1987), or a chimeric RNA-DNA analogue (Inoue et al., FEBS Lett. 215:327-30, 1987).

Antisense molecules can be generated by utilizing the Antisense Design algorithm of Integrated DNA Technologies, Inc. (1710 Commercial Park, Coralville, Iowa 52241 USA; http://www.idtdna.com/Scitools/Applications/AntiSense/Antisense.aspx. Examples of antisense nucleic acid molecules that can be utilized to decrease expression in the methods of the present invention, include, but are not limited to:

### AZIN1

GCCAATCTCCCACATTCAGC
AGCCAATCTCCCACATTCAGC
AGCCAATCTCCCACATTCAG
GCCAATCTCCCACATTCAGCT
GTGATGCTAACTCCCTTCCC
GCCAATCTCCCACATTCAG
GTTACCCTCTTCACCTCCA
AGCCAATCTCCCACATTCA
AAGCCAATCTCCCACATTCAG
AAGCCAATCTCCCACATTCA

### CENPL

GTCTTCTCCCACTTCCACAGC
TCTTCTCCCACTTCCACAGCA
TCTTCTCCCACTTCCACAGC
GTCTTCTCCCACTTCCACAG
CTTCTCCCACTTCCACAGCA
GTCTTCTCCCACTTCCACA
CTTCTCCCACTTCCACAGC
ACCCACCTCAGCCTCCCAAA
GTCCCTTTGTGTTCCTTTC
TTCTCCCACTTCCACAGCA

### SFRS18

CTTCCCGACCTACTCCTTCCT
CTTCCCGACCTACTCCTTCC
ACTTCCCGACCTACTCCTTCC
TCCCGACCTACTCCTTCCTT
TTCCCGACCTACTCCTTCCT
TCCCGACCTACTCCTTCCT
TCCCGACCTACTCCTTCCTTG
TTCCCGACCTACTCCTTCCTT
GCTGTCCTCCTTGATCCCAC
GCTGTCCTCCTTGATCCCACA

### INHBA

CCATTCTCCCTTTCCCTCCC
CCATTCTCCCTTTCCCTCCCA
ACCATTCTCCCTTTCCCTCCC
CATTCTCCCTTTCCCTCCCA
CACCATTCTCCCTTTCCCTCC
ACCATTCTCCCTTTCCCTCC
CATTCTCCCTTTCCCTCCC
CCATTCTCCCTTTCCCTCC
ATTCTCCCTTTCCCTCCCA
CACCATTCTCCCTTTCCCTC

### NAV3

GCCAGCTTCCCTTCCTTCCA
GCATCATCATCCTTCCCACCA
GCATCATCATCCTTCCCACC
ACCACTCTTTGCCCTCTTCT
CCACTCTTTGCCCTCTTCTT
TCATCCTTCCCACCATCACT
ACCACTCTTTGCCCTCTTCTT
CCACTCTTTGCCCTCTTCTTG
CCACTCTTTGCCCTCTTCT
TCATCATCCTTCCCACCATCA

### ODZ2

CACACTCCACTTCTCCTTCCC
ACACTCCACTTCTCCTTCCC
CACTCCACTTCTCCTTCCC
CACTCCACTTCTCCTTCCCG
CCACACTCCACTTCTCCTTCC
ACACTCCACTTCTCCTTCCCG
ACTCCACTTCTCCTTCCCG
ACTCCACTTCTCCTTCCCGTC
CACTCCACTTCTCCTTCCCGT
TCCACTTCTCCTTCCCGTCC

### Ostalpha

CTCCCATGTTCTGCTCACCC
CTCCCATGTTCTGCTCACCCA
TCCCATGTTCTGCTCACCCA
TCCCATGTTCTGCTCACCC
GCTCCCATGTTCTGCTCACC
CTCCCATGTTCTGCTCACC
GTTCTCTCCAGCAATCCCG
CGTTCTCTCCAGCAATCCC
CATTGTCCAAGCCATCCACCT
TCATTGTCCAAGCCATCCACC

### Ostbeta

TGTGTCTGGCTTAGGATGGG
GTGTCTGGCTTAGGATGGG
GCATCTCTTCCAGCAGCTCC
ATGACCACCACAGCTGCCAG
CTCTTAGGTTGTTTAGGCTGT
TCTGGTGGCTGCATCGTTTCT
GCATCTCTTCCAGCAGCTCCT
ATGACCACCACAGCTGCCA
GGCTGTTGTGATCCTTGGC

### PSMA4

GTGGATGTTGCGTCTCTCTG
CTTGTGGATGTTGCGTCTCTC
TGTGGATGTTGCGTCTCTCT
GTGGATGTTGCGTCTCTCT
TGTGGATGTTGCGTCTCTCTG
TTGTGGATGTTGCGTCTCTCT
GTGGATGTTGCGTCTCTCTGC
CTTGTGGATGTTGCGTCTCT
TTGTGGATGTTGCGTCTCTC
GATGTTGCGTCTCTCTGCTGC

### RHOA

TCCCACAAAGCCAACTCTACC
GTCCCACAAAGCCAACTCTAC
ACCTGCTTTCCATCCACCTC
CCTGCTTTCCATCCACCTCG
GTCCCACAAAGCCAACTCT
ACCTCTCTCACTCCATCTTT
GTCCCACAAAGCCAACTCTA
ACCTGCTTTCCATCCACCTCG
CCTGCTTTCCATCCACCTC
GTGTCCCACAAAGCCAACTCT

### RPL28

GTCATCTCATCTTCCTCCCGT
GTCATCTCATCTTCCTCCCG
GTCATCTCATCTTCCTCCC
TCATCTCATCTTCCTCCCGT
TCATCTCATCTTCCTCCCGTG
GTCCGCTTCCTCTTCACCATC
CATCTCATCTTCCTCCCGTG
AGTCCCGAGTCTCTGCTGCT
CATCTCATCTTCCTCCCGT
AGACCATCTCCCTCCCTCCA

### RPL3

GCCTCCACCACCTCCTTCTT
CCTCCACCACCTCCTTCTT
AGCCTCCACCACCTCCTTCT
AGCCTCCACCACCTCCTTCTT
GTTCCCACCACACAGCCTTTC
CCTCCACCACCTCCTTCTTG
ACAGCCTCCACCACCTCCTT
CTTCACCTTCCCACGATGCCT
GCTCTTCACCTTCCCACGA
GCTCTTCACCTTCCCACGAT

### SFRS3

AGTCTTCCCGCTTTCCTCCG
AGTCTTCCCGCTTTCCTCC
GAGTCTTCCCGCTTTCCTCC
TCTCTCTCTTCTCCTATCTCT
ATGAGTCTTCCCGCTTTCCT
TGAGTCTTCCCGCTTTCCT
CTCTCTCTTCTCCTATCTCT
TCTCTCTCTTCTCCTATCTC
GCTTGTGATTTCTCTCCCGA
GTTCCACTCTTACACGGCAGC

### SYNGR1

GCCCAGTCCCTTCTCCCATA
CCCTCCATCTCTCACCCTCT
GCTCTCCCATACCTCCCTGT
CCTCCATCTCTCACCCTCTCT
TCCCATACCTCCCTGTCCCT
GACTCCTGAACCTCTCCCTCT
ACTTCCTCCTCTTTCCCTT
CTCTCCCTCTGTGCTTGACCT
CCTCCATCTCTCACCCTCTC
GACTCCTGAACCTCTCCCTC

### TNXRD1

ATCCCTTCGATGCCCTGCCA
GTTCCATCACCGCCTACCACA
GTTCCATCACCGCCTACCAC
AGTAGCCATTTCCCTTCCT
TCCCTTCGATGCCCTGCCAA
TGTTCCATCACCGCCTACCAC
TTCCATCACCGCCTACCACA
ATCCCTTCGATGCCCTGCCAA
TCCATCACCGCCTACCACAT
TCCATCACCGCCTACCACA

Also provided are sequences comprising the antisense sequences set forth above that are not the full length mRNA for any of the genes listed in Table 1 and can be used as antisense sequences. Further provided are antisense sequences that overlap with the sequences set forth above and comprise a fragment of the above-mentioned sequences. As mentioned above, these antisense sequences are merely exemplary, as it is known to those of skill in the an that once a mRNA sequence is provided for example the mRNA sequences set forth in Table 1, it is routine to walk along the mRNA sequence to generate antisense sequences that decrease expression of for example, AZIN1. Therefore, the methods of the present invention can utilize any antisense sequence that decreases the expression of a gene set forth in Table 1.

### Morpholinos

Morpholinos are synthetic antisense oligos that can block access of other molecules to small (about 25 base) regions of ribonucleic acid (RNA). Morpholinos are often used to determine gene function using reverse genetics methods by blocking access to mRNA. Morpholinos, usually about 25 bases in length, bind to complementary sequences of RNA by standard nucleic acid base-pairing. Morpholinos do not degrade their target RNA molecules. Instead, Morpholinos act by “steric hindrance”, binding to a target sequence within an RNA and simply interfering with molecules which might otherwise interact with the RNA. Morpholinos have been used in mammals, ranging from mice to humans.

Bound to the 5′-untranslated region of messenger RNA (mRNA), Morpholinos can interfere with progression of the ribosomal initiation complex from the 5′ cap to the start codon. This prevents translation of the coding region of the targeted transcript (called “knocking down” gene expression). Morpholinos can also interfere with pre-mRNA processing steps, usually by preventing the splice-directing snRNP complexes from binding to their targets at the borders of introns on a strand of pre-RNA. Preventing U1 (at the donor site) or U2/U5 (at the polypyrimidine moiety & acceptor site) from binding can cause modified splicing, commonly leading to exclusions of exons from the mature mRNA. Targeting some splice targets results in intron inclusions, while activation of cryptic splice sites can lead to partial inclusions or exclusions. Targets of U11/U12 snRNPs can also be blocked. Splice modification can be conveniently assayed by reverse-transcriptase polymerase chain reaction (RT-PCR) and is seen as a band shift after gel electrophoresis of RT-PCR products. Methods of designing, making and utilizing morpholinos are disclosed in U.S. Pat. No. 6,867,349 which is incorporated herein by reference in its entirety.

### Small Molecules

The present invention also provides the design and synthesis of small molecules that inhibit activity of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1. One of skill in the art can search available databases to obtain three-dimensional structures of the proteins set forth herein, or three dimensional structures of the relevant domains for the proteins provided herein. For example, the skilled artisan can query the RCSB Protein Databank http://www.rcsb.org/pdb/home/home.do or http://www.rcsb.org for available three-dimensional structures. Three-dimensional structures are available for AZIN1, INHBA and RHOA. As other structures are elucidated, one of skill in the art can search this or other databases to obtain additional structural information for the genes set forth herein. In other instances, crystal structures can be generated for the same purpose. High throughput screening of compound libraries for the identification of small molecules is also contemplated by the present invention. Compound libraries are commercially available. For example, libraries can be obtained from ChemBridge Corporation (San Diego, Calif.), such as a GPCR library, a kinase targeted library (KINACore), or an ion channel library (Ion Channel Set), to name a few. Compound libraries can also be obtained from the National Institutes of Health. For example, the NIH Clinical Collection of compounds that have been used in clinical trials can also be screened. Biofocus DPI (Essex, United Kingdom) also maintains and designs compound libraries that can be purchased for screening. One of skill in the art can select a library based on the protein of interest. For example, a GPCR library can be screened to identify a compound that binds to a G protein coupled receptor. Similarly, a kinase library can be screened to identify a compound that binds to a kinase. Other libraries that target enzyme families can also be screened, depending on the type of enzyme.

Modeling techniques that allow virtual screening of compound libraries are also contemplated herein. For example, Hyperchem software (HyperCube, Inc., Gainesville, Fla.) or AutoDock software (LaJolla, Calif.) can be utilized.

Other methods of decreasing expression and/or activity include methods of interrupting or altering transcription of mRNA molecules by site-directed mutagenesis (including mutations caused by a transposon or an insertional vector). Chemical mutagenesis can also be performed in which a cell is contacted with a chemical (for example ENU) that mutagenizes nucleic acids by introducing mutations into a gene set forth in Table 1. Transcription of mRNA molecules can also be decreased by modulating a transcription factor that regulates expression of any of the genes set forth in Table 1. Radiation can also be utilized to effect mutagenesis.

### Screening Methods

The present invention provides a method of identifying a compound that binds to a gene product of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 and can decrease infection of a cell by a pathogen comprising: a) contacting a compound with a gene product of AZTN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1; b) detecting binding of the compound to the gene product; and c) associating binding with a decrease in infection by the pathogen. This method can further comprise optimizing a compound that binds the gene product in an assay, for example, a cell based assay or an in vivo assay, that determines the functional ability to decrease infection.

Further provided is a method of identifying an agent that decreases infection of a cell by a pathogen comprising: a) administering the agent to a cell containing a cellular gene encoding AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1; b) detecting the level and/or activity of the gene product produced by the cellular gene encoding AZIN1. CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1, a decrease or elimination of the gene product and/or gene product activity indicating an agent with antipathogenic activity. As mentioned above, a gene product activity can be binding between AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 and another cellular protein or nucleic acid, or binding between AZIN1. CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 and a pathogenic (i.e. non-host) protein.

Also provided is a method of identifying an agent that decreases infection in a cell by a pathogen comprising: a) administering the agent to a cell containing a cellular gene encoding AZIN1. CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1; b) associating the agent with decreasing expression or activity of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1; c) contacting the cell with a pathogen; and d) determining the level of infection, a decrease or elimination of infection indicating that the agent is an agent that decreases infection. This method can further comprise measuring the level of expression and/or activity of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1.

In the methods of the present invention, if the agent has previously been identified as an agent that decreases or inhibits the level and/or activity of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1, this can indicate a decrease in infection. A decrease in infection as compared to infection in a cell that was not contacted with the agent known to decrease or inhibit the level and/or activity of the gene product can be sufficient to identify the agent as an agent that decreases or inhibits infection.

The methods described above can be utilized to identify any agent with an activity that decreases infection, prevents infection or promotes cellular survival after infection with a pathogen(s). Therefore, the cell can be contacted with a pathogen before, or after being contacted with the agent. The cell can also be contacted concurrently with the pathogen and the agent. The agents identified utilizing these methods can be used to inhibit infection in cells either in vitro, ex vivo or in vivo.

In the methods of the present invention any cell that can be infected with a pathogen can be utilized. The cell can be prokaryotic or eukaryotic, such as a cell from an insect, fish, crustacean, mammal, bird, reptile, yeast or a bacterium, such as E. coli. The cell can be part of an organism, or part of a cell culture, such as a culture of mammalian cells or a bacterial culture. The cell can also be in a nonhuman subject thus providing in vivo screening of agents that decrease infection by a pathogen. Cells susceptible to infection are well known and can be selected based on the pathogen of interest.

The test agents or compounds used in the methods described herein can be, but are not limited to, chemicals, small molecules, inorganic molecules, organic molecules, drugs, proteins, cDNAs, large molecules, antibodies, morpholinos, triple helix molecule, peptides, siRNAs, shRNAs, miRNAs, antisense RNAs, ribozymes or any other compound. The compound can be random or from a library optimized to bind AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1. Drug libraries optimized for the proteins in the class of proteins provided herein can also be screened or tested for binding or activity. Compositions identified with the disclosed approaches can be used as lead compositions to identify other compositions having even greater antipathogenic activity. For example, chemical analogs of identified chemical entities, or variants, fragments or fusions of peptide agents, can be tested for their ability to decrease infection using the disclosed assays. Candidate agents can also be tested for safety in animals and then used for clinical trials in animals or humans.

In the methods described herein, once the cell containing a cellular gene encoding AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 has been contacted with an agent, the level of infection can be assessed by measuring an antigen or other product associated with a particular infection. For example, the level of viral infection can be measured by real-time quantitative reverse transcription-polymerase chain reaction (RT-PCR) assay (See for example, Payungporn et al. “Single step multiplex real-time RT-PCR for H5N1 influenza A virus detection.” J Virol Methods. Sep. 22, 2005; Landolt et al. “Use of real-time reverse transcriptase polymerase chain reaction assay and cell culture methods for detection of swine influenza A viruses” Am J Vet Res. 2005 January; 66(1): 119-24). If there is a decrease in infection then the composition is an effective agent that decreases infection. This decrease does not have to be complete as the decrease can be a 10%, 20%, 30%, 40%, 50%, 60%, 70%, 80%, 90%, 100% decrease or any percentage decrease in between.

In the methods set forth herein, the level of the gene product can be measured by any standard means, such as by detection with an antibody specific for the protein. The nucleic acids set forth herein and fragments thereof can be utilized as primers to amplify nucleic acid sequences, such as a gene transcript of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 by standard amplification techniques. For example, expression of a gene transcript can be quantified by real time PCR using RNA isolated from cells. A variety of PCR techniques are familiar to those skilled in the art. For a review of PCR technology, see White (1997) and the publication entitled “PCR Methods and Applications” (1991, Cold Spring Harbor Laboratory Press), which is incorporated herein by reference in its entirety for amplification methods. In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initialed. The cycles are repeated multiple times to produce an amplified fragment containing the nucleic acid sequence between the primer sites. PCR has further been described in several patents including U.S. Pat. Nos. 4,683,193, 4,683,202 and 4,963,188. Each of these publications is incorporated herein by reference in its entirety for PCR methods. One of skill in the art would know how to design and synthesize primers that amplify any of the nucleic acid sequences set forth herein or a fragment thereof.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE),6-carboxy-X-rhodamine (ROX). 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g., ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified fragment, can be analyzed by one of a number of methods known in the art. The nucleic acid can be sequenced by dideoxy or other methods. Hybridization with the sequence can also be used to determine its presence, by Southern blots, dot blots, etc.

In the methods of the present invention, the level of gene product can be compared to the level of the gene product in a control cell not contacted with the compound. The level of gene product can be compared to the level of the gene product in the same cell prior to addition of the compound. Activity or function, can be measured by any standard means, such as by enzymatic assays that measure the conversion of a substrate to a product or binding assays that measure the binding of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 to another protein, for example.

Moreover, the regulatory region of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 can be functionally linked to a reporter gene and compounds can be screened for inhibition of reporter gene expression. Such regulatory regions can be isolated from genomic sequences and identified by any characteristics observed that are characteristic for regulatory regions of the species and by their relation to the start codon for the coding region of the gene. As used herein, a reporter gene encodes a reporter protein. A reporter protein is any protein that can be specifically detected when expressed. Reporter proteins are useful for detecting or quantitating expression from expression sequences. Many reporter proteins are known to one of skill in the art. These include, but are not limited to, β-galactosidase, luciferase, and alkaline phosphatase that produce specific detectable products. Fluorescent reporter proteins can also be used, such as green fluorescent protein (GFP), cyan fluorescent protein (CFP), red fluorescent protein (RFP) and yellow fluorescent protein (YFP).

Viral infection can also be measured via cell based assays. Briefly, by way of example, cells (20.000 to 2,500,000) are infected with the desired pathogen, and the incubation continued for 3-7 days. The antiviral agent can be applied to the cells before, during, or after infection with the pathogen. Skilled practitioners can determine the amount of virus and agent administered. In some examples, several different doses of the potential therapeutic agent can be administered, to identify optimal dose ranges. Following transfection, assays are conducted to determine the resistance of the cells to infection by various agents.

For example, if analyzing viral infection, the presence of a viral antigen can be determined by using antibody specific for the viral protein then detecting the antibody. In one example, the antibody that specifically binds to the viral protein is labeled, for example with a detectable marker such as a fluorophore. In another example, the antibody is detected by using a secondary antibody containing a label. The presence of bound antibody is then detected, for example using microscopy, flow cytometry and ELISA. Similar methods can be used to monitor bacterial, protozoal, or fungal infection (except that the antibody would recognize a bacterial, protozoal, or fungal protein, respectively).

Alternatively, or in addition, the ability of the cells to survive viral infection is determined, for example, by performing a cell viability assay, such as trypan blue exclusion. Plaque assays can be utilized as well.

The amount of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 protein in a cell, can be determined by methods standard in the art for quantitating proteins in a cell, such as Western blotting, ELISA, ELISPOT, immunoprecipitation, immunofluorescence (e.g., FACS), immunohistochemistry, immunocytochemistry, etc., as well as any other method now known or later developed for quantitating protein in or produced by a cell.

The amount of an AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 nucleic acid in a cell can be determined by methods standard in the art for quantitating nucleic acid in a cell, such as in situ hybridization, quantitative PGR, RT-PCR, Taqman assay. Northern blotting, ELISPOT, dot blotting, etc., as well as any other method now known or later developed for quantitating the amount of a nucleic acid in a cell.

The ability of an antiviral agent to prevent or decrease infection by a virus, for example, any of the viruses listed above, can be assessed in an animal model. Several animal models for viral infection are known in the art. For example, mouse HIV models are disclosed in Sutton et al. (Res. Initial Treat Action, 8:22-4, 2003) and Pincus et al. (AIDS Res. Hum. Retroviruses 19:901-8, 2003); guinea pig models for Ebola infection are disclosed in Parren et al. (J. Virol 76:6408-12, 2002) and Xu et al. (Nat. Med. 4:37-42, 1998); cynomolgus monkey (Macaca fascicularis) models for influenza infection are disclosed in Kuiken et al. (Vet. Pathol 40:304-10, 2003); mouse models for herpes are disclosed in Wu et al. (Cell Host Microbe 22:5(1):84-94, 2009); pox models are disclosed in Smee et al. (Nucleosides Nucleotides Nucleic Acids 23(1-2):375-83, 2004) and in Bray et al. (J. Infect. Dis. 181 (I): 10-19); and Franciscella tularensis models are disclosed in Klimpel et al. (Vaccine 26(52): 6874-82, 2008).

Other animal models for influenza infection are also available. These include, but are not limited to, a cotton rat model disclosed by Ottolini et al. (J. Gen. Virol., 86(Pt 10): 2823-30, 2005), as well as ferret and mouse models disclosed by Maines et al. (J. Virol. 79(18): 11788-11800, 2005). One of skill in the art would know how to select an animal model for assessing the in vivo activity of an agent for its ability to decrease infection by viruses, bacteria, fungi and parasites.

Such animal models can also be used to test agents for an ability to ameliorate symptoms associated with viral infection. In addition, such animal models can be used to determine the LD₅₀ and the ED₅₀ in animal subjects, and such data can be used to determine the in vivo efficacy of potential agents. Animal models can also be used to assess antibacterial, antifungal and antiparasitic agents.

Animals of any species, including, but not limited to, birds, ferrets, cats, mice, rats, rabbits, fish (for example, zebrafish) guinea pigs, pigs, micro-pigs, goats, and non-human primates, e.g., baboons, monkeys, and chimpanzees, can be used to generate an animal model of viral infection, bacterial infection, fungal infection or parasitic infection if needed.

For example, for a model of viral infection, the appropriate animal is inoculated with the desired virus, in the presence or absence of the antiviral agent. Skilled practitioners can determine the amount of virus and agent administered. In some examples, several different doses of the potential therapeutic agent (for example, an antiviral agent) can be administered to different test subjects, to identify optimal dose ranges. The therapeutic agent can be administered before, during, or after infection with the virus. Subsequent to the treatment, animals are observed for the development of the appropriate viral infection and symptoms associated therewith. A decrease in the development of the appropriate viral infection, or symptoms associated therewith, in the presence of the agent provides evidence that the agent is a therapeutic agent that can be used to decrease or even inhibit viral infection in a subject. For example, a virus can be tested which is lethal to the animal and survival is assessed. In other examples, the weight of the animal or viral titer in the animal can be measured. Similar models and approaches can be used for bacterial, fungal and parasitic infections.

In the methods of the present invention, the level of infection can be associated with the level of gene expression and/or activity, such that a decrease or elimination of infection associated with a decrease or elimination of gene expression and/or activity indicates that the agent is effective against the pathogen. For example, the level of infection can be measured in a cell after administration of siRNA that is known to inhibit AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1. If there is a decrease in infection then the siRNA is an effective agent that decreases infection. This decrease does not have to be complete as the decrease can be a 10%, 20%, 30%, 40%, 50%, 60%, 70%, 80%, 90%, 100% decrease or any percentage decrease in between. In the event that the compound is not known to decrease AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 expression and/or activity, the level of expression and/or activity of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 can be measured utilizing the methods set forth above and associated with the level of infection. By correlating a decrease in AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 expression with a decrease in infection, one of skill in the an can confirm that a decrease in infection is effected by a decrease in AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 expression and/or activity. Similarly, the level of infection can be measured in a cell, utilizing the methods set forth above and known in the art, after administration of a chemical, small molecule, drug, protein, cDNA, antibody, shRNA, miRNA, morpholino, antisense RNA, ribozyme or any other compound. If there is a decrease in infection, then the chemical, small molecule, drug, protein, cDNA, antibody, shRNA, miRNA, morpholino, antisense RNA, ribozyme or any other compound is an effective antpathogenic agent.

The AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 genes and nucleic acids of the invention can also be used in polynucleotide arrays. Polynucleotide arrays provide a high throughput technique that can assay a large number of polynucleotide sequences in a single sample. This technology can be used, for example, to identify samples with reduced expression of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 as compared to a control sample. This technology can also be utilized to determine the effects of reduced expression of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 on other genes. In this way, one of skill in the art can identify genes that are unregulated or downregulated upon reduction of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 expression. Similarly, one of skill in the art can identify genes that are upregulated or downregulated upon increased expression of AZIN1, CENPL, C6orf111 (SFRS18), INHBA, NAV3, ODZ2, Ost-alpha, Ost-beta, PSMA4, RHOA, RPL28, RPL3, SFRS3, SYNCR1 or TXNRD1. This allows identification of other genes that are upregulated or downregulated upon modulation of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 expression that can be targets for therapy, such as antiviral therapy, antibacterial therapy, antiparasitic therapy or antifungal therapy.

To create arrays, single-stranded polynucleotide probes can be spotted onto a substrate in a two-dimensional matrix or array. Each single-stranded polynucleotide probe can comprise at least 6, 7, 8, 9, 10, 11, 12, 13, 14, 15, 16, 17, 18, 19, 20, 25, or 30 or more contiguous nucleotides selected from nucleotide sequences set forth under GenBank Accession Nos. herein and other nucleic acid sequences that would be selected by one of skill in the art depending on what genes, in addition to AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 are being analyzed.

The array can also be a microarray that includes probes to different polymorphic alleles of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1. A polymorphism exists when two or more versions of a nucleic acid sequence exist within a population of subjects. For example, a polymorphic nucleic acid can be one where the most common allele has a frequency of 99% or less. Different alleles can be identified according to differences in nucleic acid sequences, and genetic variations occurring in more than 1% of a population (which is the commonly accepted frequency for defining polymorphism) are useful polymorphisms for certain applications.

The allelic frequency (the proportion of all allele nucleic acids within a population that are of a specified type) can be determined by directly counting or estimating the number and type of alleles within a population. Polymorphisms and methods of determining allelic frequencies are discussed in Haiti. D. L. and Clark, A. G., Principles of Population Genetics, Third Edition (Sinauer Associates, Inc., Sunderland Mass., 1997), particularly in chapters 1 and 2.

These microarrays can be utilized to detect polymorphic alleles in samples from subjects. Such alleles may indicate that a subject is more susceptible to infection or less susceptible to infection. For example, since the present invention shows that a disruption in AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 expression results in decreased viral infection, such microarrays can be utilized to detect polymorphic versions of AZIN1. CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 that result in decreased gene expression and/or decreased activity of the gene product to identify subjects that are less susceptible to viral infection. In addition, the existence of an allele associated with decreased expression in a healthy individual can be used to determine which genes are likely to have the least side effects if the gene product is inhibited or bound or may be selected for in commercial animals and bred into the population.

The substrate can be any substrate to which polynucleotide probes can be attached, including but not limited to glass, nitrocellulose, silicon, and nylon. Polynucleotide probes can be bound to the substrate by either covalent bonds or by non-specific interactions, such as hydrophobic interactions. Techniques for constructing arrays and methods of using these arrays are described in EP No. 0 799 897; PCT No. WO 97/29212; PCT No. WO 97/27317; EP No. 0 785 280: PCT No. WO 97/02357; U.S. Pat. Nos. 5,593,839; 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734. Commercially available polynucleotide arrays, such as Affymetrix GeneChip™, can also be used. Use of the GeneChip™ to detect gene expression is described, for example, in Lockhart et al. Nature Biotechnology 14:1675 (1996): Chee et al. Science 274:610 (1996); Hacia et al. Nature Genetics 14:441, 1996; and Kozal et al. Nature Medicine 2:753, 1996.

### Pharmaceutical Compositions and Modes of Administration

The present invention provides a method of decreasing infection by a pathogen in a subject by decreasing the expression or activity of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 in the subject said method comprising administering to the subject an effective amount of a composition that decreases the expression or activity of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 in the subject. The composition can comprise one or more of, a chemical, a compound, a small molecule, an inorganic molecule, an organic molecule, a drug, a protein, a cDNA, a peptide, an antibody, a morpholino, a triple helix molecule, an siRNA, an shRNAs, an miRNA, an antisense nucleic acid or a ribozyme that decreases the expression or activity of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1. The composition can be administered before or after infection. The decrease in infection in a subject need not be complete as this decrease can be a 10%, 20%, 30%, 40%, 50%, 60%, 70%, 80%, 90%, 100% or any other percentage decrease in between as long as a decrease occurs. This decrease can be correlated with amelioration of symptoms associated with infection. These compositions can be administered to a subject alone or in combination with other therapeutic agents described herein, such as anti-viral compounds, antibacterial agents, antifungal agents, antiparasitic agents, anti-inflammatory agents, anti-cancer agents, etc. Examples of viral infections, bacterial infections, fungal infections parasitic infections are set forth above. The compounds set forth herein or identified by the screening methods set forth herein can be administered to a subject to decrease infection by any pathogen or infectious agent set forth herein. Any of the compounds set forth herein or identified by the screening methods of the present invention can also be administered to a subject to decrease infection by any pathogen, now known or later discovered in which AZIN1, CENPL, C6orf111 (SFRS18), INHBA, NAV3, ODZ2, Ost-alpha, Ost-beta, PSMA4, RHOA, RPL28, RPL3. SFRS3, SYNGR1 or TXNRD1 is involved.

Various delivery systems for administering the therapies disclosed herein are known, and include encapsulation in liposomes, microparticles, microcapsules, expression by recombinant cells, receptor-mediated endocytosis (Wu and Wu, J. Biol. Chem. 1987, 262:4429-32), and construction of therapeutic nucleic acids as part of a retroviral or other vector. Methods of introduction include, but are not limited to, mucosal, topical, intradermal, intrathecal, intratracheal, via nebulizer, via inhalation, intramuscular, intraperitoneal, vaginal, rectal, intravenous, subcutaneous, intranasal, and oral routes. The compounds can be administered by any convenient route, for example by infusion or bolus injection, by absorption through epithelial or mucocutaneous linings (for example, oral mucosa, rectal, vaginal and intestinal mucosa, etc.) and can be administered together with other biologically active agents. Administration can be systemic or local. Pharmaceutical compositions can be delivered locally to the area in need of treatment, for example by topical application or local injection.

Pharmaceutical compositions are disclosed that include a therapeutically effective amount of a RNA, DNA, antisense molecule, ribozyme, siRNA, shRNA molecule, miRNA molecule, drug, protein, small molecule, peptide inorganic molecule, organic molecule, antibody or other therapeutic agent, alone or with a pharmaceutically acceptable carrier. Furthermore, the pharmaceutical compositions or methods of treatment can be administered in combination with (such as before, during, or following) other therapeutic treatments, such as other antiviral agents, antibacterial agents, antifungal agents and antiparasitic agents.

For all of the administration methods disclosed herein, each method can optionally comprise the step of diagnosing a subject with an infection or diagnosing a subject in need of prophylaxis or prevention of infection.

### Delivery Systems

The pharmaceutically acceptable carriers useful herein are conventional. Remington's Pharmaceutical Sciences, by Martin, Mack Publishing Co., Easton, Pa., 15th Edition (1975), describes compositions and formulations suitable for pharmaceutical delivery of the therapeutic agents herein disclosed. In general, the nature of the carrier will depend on the mode of administration being employed. For instance, parenteral formulations usually include injectable fluids that include pharmaceutically and physiologically acceptable fluids such as water, physiological saline, balanced salt solutions, aqueous dextrose, sesame oil, glycerol, ethanol, combinations thereof, or the like, as a vehicle. The carrier and composition can be sterile, and the formulation suits the mode of administration. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The composition can be a liquid solution, suspension, emulsion, tablet, pill, capsule, sustained release formulation, or powder. For solid compositions (for example powder, pill, tablet or capsule forms), conventional non-toxic solid carriers can include, for example, pharmaceutical grades of mannitol, lactose, starch, sodium saccharine, cellulose, magnesium carbonate, or magnesium stearate. The composition can be formulated as a suppository, with traditional binders and carriers such as triglycerides.

Embodiments of the disclosure including medicaments can be prepared with conventional pharmaceutically acceptable carriers, adjuvants and counterions as would be known to those of skill in the art.

The amount of therapeutic agent effective in decreasing or inhibiting infection can depend on the nature of the pathogen and its associated disorder or condition, and can be determined by standard clinical techniques. Therefore, these amounts will vary depending on the type of virus, bacteria, fungus, parasite or other pathogen. For example, the dosage can be anywhere from 0.01 mg/kg to 100 mg/kg. Multiple dosages can also be administered depending on the type of pathogen, and the subject's condition. In addition, in vitro assays can be employed to identify optimal dosage ranges. The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems.

The disclosure also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration. Instructions for use of the composition can also be included.

In an example in which a nucleic acid is employed to reduce infection, such as an antisense or siRNA molecule, the nucleic acid can be delivered intracellularly (for example by expression from a nucleic acid vector or by receptor-mediated mechanisms), or by an appropriate nucleic acid expression vector which is administered so that it becomes intracellular, for example by use of a retroviral vector (see U.S. Pat. No. 4,980,286), or by direct injection, or by use of microparticle bombardment (such as a gene gun; Biolistic, Dupont), or coating with lipids or cell-surface receptors or transfecting agents, or by administering it in linkage to a homeobox-like peptide which is known to enter the nucleus (for example Joliot et al., Proc. Natl. Acad. Sci. USA 1991, 88:1864-8). siRNA carriers also include, polyethylene glycol (PEG), PEG-liposomes, branched carriers composed of histidine and lysine (HK polymers), chitosan-thiamine pyrophosphate carriers, surfactants (for example. Survanta and Infasurf), nanochitosan carriers, and D5W solution. The present disclosure includes all forms of nucleic acid delivery, including synthetic oligos, naked DNA, plasmid and viral delivery, integrated into the genome or not.

As mentioned above, vector delivery can be via a viral system, such as a retroviral vector system which can package a recombinant retroviral genome (see e.g., Pastan et al., Proc. Natl. Acad. Sci. U.S. 85:4486, 1988; Miller et al., Mol. Cell. Biol. 6:2895, 1986). The recombinant retrovirus can then be used to infect and thereby deliver to the infected cells a nucleic acid, for example an antisense molecule or siRNA. The exact method of introducing the altered nucleic acid into mammalian cells is, of course, not limited to the use of retroviral vectors. Other techniques are widely available for this procedure including the use of adenoviral vectors (Mitani et al. Hum. Gene Ther. 5:941-948, 1994), adeno-associated viral (AAV) vectors (Goodman et al. Blood 84:1492-1500, 1994), lentiviral vectors (Naidini et al. Science 272:263-267, 1996), and pseudotyped retroviral vectors (Agrawal et al., Exper. Hematol. 24:738-747, 1996). Other nonpathogenic vector systems such as the foamy virus vector can also be utilized (Park et al. “Inhibition of simian immunodeficiency virus by foamy virus vectors expressing siRNAs.” Virology. 2005 Sep. 20). It is also possible to deliver short hairpin RNAs (shRNAs) via vector delivery systems in order to inhibit gene expression (See Pichler et al. “In vivo RNA interference-mediated ablation of MDR1 P-glycoprotein.” Clin Cancer Res. 2005 Jun. 15; 11(12):4487-94; Lee et al. “Specific inhibition of HIV-1 replication by short hairpin RNAs targeting human cyclin T1 without inducing apoptosis.” FEBS Lett. 2005 Jun. 6; 579(14):3100-6.).

Physical transduction techniques can also be used, such as liposome delivery and receptor-mediated and other endocytosis mechanisms (sec, for example, Schwartzenberger et al. Blood 87:472-478, 1996) to name a few examples. This invention can be used in conjunction with any of these or other commonly used gene transfer methods.

### Transgenic Cells and Non-Human Mammals

The present invention also provides a non-human transgenic mammal comprising a functional deletion of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1, wherein the mammal has decreased susceptibility to infection by a pathogen, such as a virus, a bacterium, a fungus or a parasite. Exemplary transgenic non-human mammals include, but are not limited to, ferrets, fish, guinea piags, chinchilla, mice, monkeys, rabbits, rats, chickens, cows, and pigs. Such knock-out animals are useful for reducing the transmission of viruses from animals to humans and for further validating a target. In the transgenic animals of the present invention one or both alleles of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 can be functionally deleted.

By “decreased susceptibility” is meant that the animal is less susceptible to infection or experiences decreased infection by a pathogen as compared to an animal that does not have one or both alleles of a AZIN1. CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 functionally deleted. The animal does not have to be completely resistant to the pathogen. For example, the animal can be 10%, 20%, 30%, 40%, 50%, 60%, 70%, 80%, 90%, 100% or any percentage in between less susceptible to infection by a pathogen as compared to an animal that does not have a functional deletion of AZIN1. CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1. Furthermore, decreasing infection or decreasing susceptibility to infection includes decreasing entry, replication, pathogenesis, insertion, lysis, or other steps in the replication strategy of a virus or other pathogen into a cell or subject, or combinations thereof.

Therefore, the present invention provides a non-human transgenic mammal comprising a functional deletion of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1, wherein the mammal has decreased susceptibility to infection by a pathogen, such as a virus, a bacterium, a parasite or a fungus. A functional deletion is a mutation, partial or complete deletion, insertion, or other variation made to a gene sequence that inhibits production of the gene product or renders a gene product that is not completely functional or non-functional. Functional deletions can be made by insertional mutagenesis (for example via insertion of a transposon or insertional vector), by site directed mutagenesis, via chemical mutagenesis, via radiation or any other method now known or developed in the future that results in a transgenic animal with a functional deletion of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1.

Alternatively, a nucleic acid sequence such as siRNA, a morpholino or another agent that interferes with AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 mRNA expression can be delivered. The expression of the sequence used to knock-out or functionally delete the desired gene can be regulated by an appropriate promoter sequence. For example, constitutive promoters can be used to ensure that the animal does not express the functionally deleted gene. In contrast, an inducible promoter can be used to control when the transgenic animal does or does not express the gene of interest. Exemplary inducible promoters include tissue-specific promoters and promoters responsive or unresponsive to a particular stimulus (such as light, oxygen, chemical concentration, such as a tetracycline inducible promoter).

The transgenic animals of the present invention that comprise a functionally deleted AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 gene can be examined during exposure to various pathogens. Comparison data can provide insight into the life cycles of pathogens. Moreover, knockout animals or functionally deleted (such as birds or pigs) that are otherwise susceptible to an infection (for example influenza) can be made to resist infection, conferred by disruption of the gene. If disruption of the gene in the transgenic animal results in an increased resistance to infection, these transgenic animals can be bred to establish flocks or herds that are less susceptible to infection.

Transgenic animals, including methods of making and using transgenic animals, are described in various patents and publications, such as WO 01/43540; WO 02/19811; U.S. Pub. Nos: 2001-0044937 and 2002-0066117; and U.S. Pat. Nos. 5,859,308; 6,281,408; and 6,376,743; and the references cited therein.

The transgenic animals of this invention also include conditional gene knockdown animals produced, for example, by utilizing the SIRIUS-Cre system that combines siRNA for specific gene-knockdown, Cre-IoxP for tissue-specific expression and tetracycline-on for inducible expression. Mating two parental lines that contain a specific siRNA of interest gene and tissue-specific recombinase under tetracycline control can generate these animals. See Chang et al. “Using siRNA Technique to Generate Transgenic Animals with Spatiotemporal and Conditional Gene Knockdown.” American Journal of Pathology 165: 1535-1541 (2004) which is hereby incorporated in its entirety by this reference regarding production of conditional gene knockdown animals.

The present invention also provides cells including an altered or disrupted AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 that are resistant to infection by a pathogen. These cells can be in vitro, ex vivo or in vivo cells and can have one or both alleles altered. These cells can also be obtained from the transgenic animals of the present invention. Such cells therefore include cells having decreased susceptibility to a virus or any of the other pathogens described herein, including bacteria, parasites and fungi.

Since the genes set forth herein are involved in viral infection, also provided herein are methods of overexpressing any of the genes set forth in Table 1 in host cells. Overexpression of these genes can provide cells that increase the amount of virus produced by the cell, thus allowing more efficient production of viruses. Also provided is the overexpression of the genes set forth herein in avian eggs, for example, in chicken eggs.

Methods of screening agents, such as a chemical, a compound, a small or large molecule, an organic molecule, an inorganic molecule, a peptide, a drug, a protein, a cDNA, an antibody, a morpholino, a triple helix molecule, an siRNA, an shRNAs, an miRNA, an antisense nucleic acid or a ribozyme set forth using the transgenic animals described herein are also provided.

### Screening for Resistance to Infection

Also provided herein are methods of screening host subjects for resistance to infection by characterizing a nucleotide sequence of a host AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 nucleic acid or corresponding amino acid sequence. The nucleic acid or amino acid sequence of a subject can be isolated, sequenced, and compared to the wildtype sequence for AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1. The greater the similarity between that subject's AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 nucleic acid and the wildtype sequence, the more susceptible that person is to infection, while a decrease in similarity between that subject's AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 nucleic acid and the wildtype sequence, the more resistant that subject can be to infection. Such screens can be performed for any AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 host nucleic acid or the corresponding amino acid sequence in any species.

Assessing the genetic characteristics of a population can provide information about the susceptibility or resistance of that population to viral infection. For example, polymorphic analysis of alleles in a particular human population, such as the population of a particular city or geographic area, can indicate how susceptible that population b to infection. A higher percentage of alleles substantially similar to wild-type AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 can indicate that the population is more susceptible to infection, while a large number of polymorphic alleles that are substantially different than wild-type AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 sequences can indicate that a population is more resistant to infection. Such information can be used, for example, in making public health decisions about vaccinating susceptible populations.

The present invention also provides a method of screening a cell for a variant form of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1. A variant can be a gene with a functional deletion, mutation or alteration in the gene such that the amount or activity of the gene product is altered. These cells containing a variant form of a gene can be contacted with a pathogen to determine if cells comprising a naturally occurring variant of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 differ in their resistance to infection. For example, cells from an animal, for example, a chicken, can be screened for a variant form of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1. If a naturally occurring variant is found and chickens possessing a variant form of the gene in their genome are less susceptible to infection, these chickens can be selectively bred to establish flocks that are resistant to infection. By utilizing these methods, flocks of chickens that are resistant to avian flu or other pathogens can be established. Similarly, other animals can be screened for a variant form of a gene AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1. If a naturally occurring variant is found and animals possessing a variant form of the gene in their genome are less susceptible to infection, these animals can be selectively bred to establish populations that are resistant to infection. These animals include, but are not limited to, cats, dogs, livestock (for example, cattle, horses, pigs, sheep, goats, etc.), laboratory animals (for example, mouse, monkey, rabbit, rat, gerbil, guinea pig, etc.) and avian species (for example, flocks of chickens, geese, turkeys, ducks, pheasants, pigeons, doves etc.). Therefore, the present application provides populations of animals that comprise a naturally occurring variant of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 that results in decreased susceptibility to viral infection, thus providing populations of animals that are less susceptible to viral infection. Similarly, if a naturally occurring variant is found and animals possessing a variant form of the gene in their genome are less susceptible to bacterial, parasitic or fungal infection, these animals can be selectively bred to establish populations that are resistant to bacterial parasitic or fungal infection.

Also provided is a method of making a compound that decreases infection of a cell by a pathogen, comprising: a) synthesizing a compound; b) administering the compound to a cell containing a cellular gene encoding AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1; c) contacting the cell with an infectious pathogen;

d) determining the level of infection, a decrease or elimination of infection indicating that the agent is an agent that decreases infection; e) associating the agent with decreasing expression or activity of AZIN1. CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1.

This method can further comprise making the association by measuring the level of expression and/or activity of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1.

Further provided is a method of making a compound that decreases infection in a cell by a pathogen, comprising: a) optimizing a compound to bind AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1; b) administering the compound to a cell containing a cellular gene encoding AZIN1. CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1; c) contacting the cell with an infectious pathogen; d) determining the level of infection, a decrease or elimination of infection indicating the making of a compound that decreases infection in a cell by a pathogen. This method can further comprise making a compound that decreases infection in a cell by a pathogen comprising synthesizing therapeutic quantities of the compound made.

The present invention also provides a method of synthesizing a compound that binds to a gene product of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 and decreases infection by a pathogen comprising: a) contacting a library of compounds with a gene product of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1; b) associating binding with a decrease in infection; and c) synthesizing derivatives of the compounds from the library that bind to the gene product of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1.

Further provided is a business method to reduce the cost of discovery of drugs that can reduce infection by a pathogen comprising: a) screening, outside of the United States, for drugs that reduce infection by binding to or reducing the function of a gene product of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1; and b) importing active drugs into the United States.

Also provided is a method of making drugs comprising directing the synthesis of drugs that reduce infection by binding to or reducing the function of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1 or gene product of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1.

The following examples are put forth so as to provide those of ordinary skill in the art with a complete disclosure and description of how the antibodies, polypeptides, nucleic acids, compositions, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc), but some errors and deviations should be accounted for.

### EXAMPLES

Following infection with the U3NeoSV1 retrovirus gene trap shuttle vector, libraries of mutagenized Veto cells were isolated in which each clone contained a single gene disrupted by provirus integration. Gene entrapment was performed essentially as described in U.S. Pat. No. 6,448,000 and U.S. Pat. No. 6,777,177. The entrapment libraries were infected with cowpox, and virus-resistant clones were selected as described below.

Four days prior to infection, Vero gene trap library cells were thawed and centrifuged at 700 rpm for 5 minutes to pellet the cells. The supernatant was discarded. The cells were resuspended in complete growth medium ⅓ of the aliquot of cells was seeded into 6 T150 flasks with re-closeable lids. Cells were allowed to grow for 4 days at 37° C. in 5% CO₂ or until the cells were 70-100% confluent. On the day of infection, the medium in the T150 flasks was replaced with 19 mLs of fresh complete growth medium immediately before infecting the cells. A 200 μL aliquot of cowpox virus from the −80% freezer at 4° C. was thawed for 30 minutes and then diluted into 6.5 mLs of complete growth medium. Approximately 1.1 mLs of diluted virus was added to each of the 6 T150 flasks containing Vera gene trap library cells. The cells were incubated at 37° C. 5% CO₂ for 1 hour. The medium from the flasks was discarded and replaced with 20 mLs of fresh complete growth medium to remove the inoculum. The cells were incubated al 37° C., 5% CO₂.

Cells were incubated for 3-4 days until the cells were approximately 75% dead. After cells were approximately >75% dead, the medium was changed daily until day 7 post-infection. The medium was changed on days 10, 14, 17, 21, etc post-infection (following this pattern of days): Cowpox-resistant colonies were observed about 2 weeks post-infection under the microscope. When colonies appeared which were visible with the naked eye, they were circled on the bottom side of the flasks. These colonies were looked at under the microscope to determine which colonies are (A) from unhealthy/dying cells or are (B) actually two colonies very close together.

A 24-well plate with 800 μL of complete growth medium in as many wells as there are marked colonies were prepared. Resistant cells were trypsinized and cells from each cowpox-resistant clone were transferred to a single well of the 24 well plate (already containing 800 μL of complete growth medium). This process was repeated for each colony. After all colonies were added to a 24-well plate, the medium was mixed in 4 or 6 wells of the 24-well plate with a 1000 μL multichannel pipette set on 200 μL, and 200 μL of cell mixture was distributed into duplicate wells of a separate 24-well plate (already containing 500 μL of complete growth medium per well). This allowed clones to be tested for resistance, and expansion of uninfected clones for subsequent cryopreservation and trapped gene identification. DNA from these clones was prepared and sequenced.

### Identification of Genes Disrupted in Cowpox-Resistant Clones

The U3NeoSV1 gene trap vector contains a plasmid origin of replication and ampicillin resistance gene; thus, regions of genomic DNA adjacent to the targeting vector were readily cloned by plasmid rescue and sequence. The flanking sequences were compared to the nucleic acid databases to identify candidate cellular genes that confer resistance to lytic infection by cowpox virus when altered by gene entrapment. These genes are listed in Table 1.

Throughout this application, various publications are referenced. The disclosures of these publications in their entireties are hereby incorporated by reference into this application in order to more fully describe the state of the art to which this invention pertains.

### Anisomycin

Since Inhibin A, Ost-alpha and Ost-beta are genes associated with the estrogen/androgen signaling pathway, DHEA and related sterols were tested for their ability to inhibit vaccinia virus infection. As shown in Table 2, DHEA, 17-B-Estradiol, BADGE, a combination of BADGE and Tamoxifen, and a combination of DHEA and Tamoxifen inhibited infection with vaccinia virus.

Anisomycin, which mimics a function of DHEA, i.e., phosphorylation of ERK, was tested. 36 hours after infection of VERO and HELA cells in the presence of 1 uM anisomycin, there is no infection and the cells were healthy. With DHEA, the virus usually starts to spread after day 2 if the cells are soil alive. Thus anisomycin at a concentration that affects signaling pathways and not protein synthesis inhibited vaccinia virus infection. These data were reproduced with infection of RIE-1 cells with herpes simplex virus. A virus construct that expresses beta-galatosidase was used as an immediate early viral gene. When the gene is expressed and translated, a substrate turns blue which can be visualized using a standard light microscope. RIE-1 control cells in 18 h post herpes simplex infection express the reporter gene, whereas cells treated with 100 nM of anisomycin did not express immediate early herpes simplex genes. Anisomycin was effective in blocking infection, but this effect was reversed with U0126 (an MEK inhibitor). Upon addition of SP600125 (a JNK inhibitor) in the presence of anisomycin, the antiviral effect of anisomycin was not reversed and the cells were still resistant to infection. Therefore, the effects associated with anisomycin is likely not related to protein synthesis, since it effects could not be reversed upon addition of an MEK inhibitor.

TABLE 2
Effects of inhibitors/agonists of estrogen signaling
on vaccinia virus infection

| | | | % |
|---|---|---|---|
| | Effective | Effective [uM] | Effectiveness |
| DHEA | ✓ | 30 | 99 |
| DHEAS | X | NA | NA |
| 17-B Estradiol | ✓ | 100 | 50 |
| BADGE | ✓ | 50-100 | 75 |
| TAMOXIFEN | X | NA | NA |
| TAB | | | |
| TAMOXIFEN | X | NA | NA |
| PDR | | | |
| PROGESTERONE | X | NA | NA |
| PREGNENOLONE | X | NA | NA |
| CORTISOL | X | NA | NA |
| PROSTAGLAND | X | NA | NA |
| E2 | | | |
| BADGE&TAMOX | ✓ | 30 EACH | >75 |
| ESTRONE | X | NA | NA |
| ESTRIOL | X | NA | NA |
| ANISOMYCIN | ✓ | 1 | 100 |
| DHEA & TAMOX | ✓ | 30 EACH | 90 |
| U0126 | X | | |
| ANISOW/U0126 | X | | |

1. A method of decreasing infection in a cell by a pathogen comprising decreasing expression or activity of AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1.

2. The method of claim 1, wherein infection is decreased by decreasing the replication of the pathogen.

3. The method of claim 1, wherein the pathogen is a virus.

4-20. (canceled)

21. A cell comprising an altered or disrupted nucleic acid encoding AZIN1, CENPL, C6orf111 (SFRS18), INHBA, NAV3, ODZ2, Ost-alpha, Ost-beta, PSMA4, RHOA, RPL28, RPL3, SFRS3, SYNGR1 or TXNRD1, wherein the cell has decreased susceptibility to infection by a pathogen.

22. The cell of claim 21, wherein the pathogen is a virus and the cell is infected with a virus.

23-57. (canceled)

58. The method of claim 3, wherein the virus is a respiratory virus.

59. The method of claim 58, wherein the respiratory virus is a picornavirus, an orthomyxovirus, a paramyxovirus, a coronavirus, or an adenovirus.

60. The method of claim 59, wherein the respiratory virus is selected from the group consisting of influenza virus, a pox virus, parainfluenza virus, adenovirus, measles, rhinovirus, and RSV.

61. The cell of claim 22, wherein the virus is a respiratory virus.

62. The cell of claim 61, wherein the respiratory virus is a picornavirus, an orthomyxovirus, a paramyxovirus, a coronavirus, or an adenovirus.

63. The cell of claim 62, wherein the respiratory virus is selected from the group consisting of influenza virus, a pox virus, parainfluenza virus, adenovirus, measles, rhinovirus, and RSV.
